(19) United States
(12) Patent Application Publication
(10) Pub. No.: US 2008/0226724 A1
(43) Pub. Date: Sep. 18, 2008

(54) Prevention of hydrogel viscosity loss

(75) Inventors: Junyan Andrea Ji, Foster City, CA (US); Erika Ingham, Berkeley, CA (US); Yuchang John Wang, Burlingame, CA (US)

Correspondence Address: QUINE INTELLECTUAL PROPERTY LAW GROUP, P.C., P O BOX 458, ALAMEDA, CA, 94501, US

(73) Assignee: Genentech, Inc., South San Francisco, CA (US)

(21) Appl. No.: 12/011,027

(22) Filed: Jan. 22, 2008

Related U.S. Application Data
(60) Provisional application No. 60/881,260, filed on Jan. 19, 2007.

(51) Int. Cl. A61K 9/10; A61K 38/00; B01J 13/00

(52) U.S. Cl. 424/486; 516/106; 514/12

(57) ABSTRACT

The present invention comprises methods and compositions for decreasing or preventing viscosity loss in hydrogels.

### FIELD OF THE INVENTION

The current invention relates to the field of hydrogels. More specifically, the present invention provides methods for stabilizing hydrogel viscosity, especially for hydrogels that are autoclave sterilized and/or stored for extended periods of time, as well as providing compositions of such stabilized hydrogels.

### BACKGROUND OF THE INVENTION

Hydrogels of various compositions are widely used in myriad applications from paints to pharmaceuticals. For example, promising advances in wound treatments utilize a number of different types of hydrogels. Hydrogels comprising cellulose and cellulose derivatives are especially common in many applications. However, a drawback to some uses of hydrogels, and cellulose based hydrogels in particular, is that such gels lose their viscosity under certain conditions. For example, many cellulose based hydrogels lose viscosity when autoclave sterilized, thus, hampering their use in pharmaceutical compositions where sterility can be of importance (e.g., when hydrogels are used in contact with an open wound). Additionally, storage of hydrogels can allow the reaction of hydrogel polymers with free radicals, which can also lead to viscosity loss. Such loss is often exacerbated by elevated temperatures, and is problematic for hydrogels that need to be stored or transported. Prior attempts to prevent or reduce hydrogel viscosity loss have been unsuccessful and/or impractical.

Thus, there is a continuing need for better, more economical hydrogel compositions and for methods to ensure proper viscosity for hydrogels, especially in regard to hydrogels to be autoclave sterilized and/or those that are to be stored for various time periods (e.g., for certain pharmaceutical applications). The current invention provides these and other benefits which will be apparent upon examination.

### SUMMARY OF THE INVENTION

In various aspects herein, the invention comprises methods (e.g., pharmaceutical methods) for decreasing or preventing viscosity loss (e.g., preserving or stabilizing viscosity, decreasing loss in viscosity, etc.) in various hydrogel formulations. Viscosity loss can arise from reaction between free radicals (e.g., hydrogen peroxide) and hydrogel components such as the hydrogel polymers, and/or from autoclaving or heating of the hydrogel, as well as from other environmental conditions. In various embodiments, the methods comprise providing a hydrogel solution (or mixture, colloid, suspension, or the like) having one or more metal ion chelators and/or one or more radical scavenging moieties. In certain embodiments, the hydrogel is autoclave treated (e.g., sterilized) or treatable (e.g., sterilizable). Also in certain embodiments in which the hydrogel is autoclave treated, the chelator (if present in the embodiment) is added to the hydrogel solution before the hydrogel is autoclave sterilized, while the radical scavenging moiety (if present in the embodiment) is added to the solution after the solution is autoclave sterilized. In other embodiments, the hydrogel is not autoclave treated.

The methods herein optionally comprise use of any of a variety of polymer molecules capable of forming a hydrogel, such as, but not limited to: polysaccharide molecules, cellulose molecules, cellulose derivative molecules, methylcellulose molecules, hydroxypropyl methylcellulose (hypromellose) molecules, carboxymethyl cellulose molecules, hydroxypropyl cellulose molecules, hydroxyethyl cellulose molecules, hyaluronic acid molecules, Carbopol® molecules (Noveon, Cleveland, Ohio) and their derivatives, alginate, sodium hyaluronate, gellan, carrageenan, pectin, gelatin, polyvinyl pyrrolidone, poloxamer, dextran, etc. The concentration of hydrogels herein can be quantified by percent polymer molecule to hydrogel solution (weight/volume). The concentration of polymer molecule for cellulose/cellulose derivatives (e.g., methylcellulose, hydroxypropyl methylcellulose, hydroxyethylcellulose, Methocel® A4M, grade HEC hydroxyethyl cellulose, or Methocel® E4M) in the hydrogels can comprise, e.g., from about 1% to about 6% polymer molecule to hydrogel solution, from about 1.25% to about 5.5% polymer molecule to hydrogel solution, from about 1.5% to about 5.0% polymer molecule to hydrogel solution, from about 1.75% to about 4.5% polymer molecule to hydrogel solution, from about 2% to about 4% polymer molecule to hydrogel solution, from about 2.25% to about 4% polymer molecule to hydrogel solution, from about 2.5% to about 3.5% polymer molecule to hydrogel solution, or about 3% polymer molecule to hydrogel solution. It will be understood, however, that specific recitation of polymer concentrations in the various hydrogels herein should not necessarily be taken as limiting and that the methods (and the compositions) herein can also comprise lower or higher concentrations of hydrogel polymers in the hydrogels (e.g., depending upon the desired viscosity and upon the specific polymer used, etc.). Furthermore, for hydrogels based on non-cellulose/non-cellulose derivative polymers, the polymer concentration can be lower or higher than the polymer concentration for cellulose/cellulose derivative hydrogels. For example, hydrogels based on poloxamer can comprise, e.g., from about 15% to about 35%, from about 20% to about 30%, or about 25% polymer molecule to hydrogel solution, while pectin based hydrogels can comprise, e.g., about 3% to about 6%, or about 5% polymer to hydrogel solution and carrageenan based hydrogels can comprise from about 2% to about 10% polymer per hydrogel solution. Furthermore, hydrogels based on polyvinyl pyrrolidone can comprise, e.g., about 10% or more polymer per hydrogel solution and hydrogels based on dextran can comprise, e.g., from about 5% to about 10% polymer per hydrogel solution, e.g., depending on the molecular weight of the dextran. Those of skill in the art will be familiar with a range of appropriate polymer concentrations for numerous different hydrogel constructions.

Additionally, in the various methods, the chelator is typically an autoclave stable chelator (or temperature stable chelator) such as, but not limited to, an aminopolycarboxylate, EDTA, NTA, EDDS, EGTA, PDTA, or DTPA. In many embodiments, the hydrogel comprises from about 50 to about 2000 ppm chelator, from about 100 to about 1500 ppm chelator, from about 200 to about 1000 ppm chelator, from about 300 to about 500 ppm chelator, or about 400 ppm chelator (e.g., EDTA), etc.

Furthermore, the radical scavenging moieties in the methods of the invention can comprise, but are not limited to, methionine or methionine derivatives, or short peptides comprising one or more methionine residues or methionine derivatives, etc. In embodiments comprising methionine, the hydrogel solutions can comprise from about 0.01 to about 10 mg/ml, from about 0.05 to about 10 mg/ml, from about 0.1 to about 10 mg/ml, from about 0.15 to about 10 mg/ml, from about 0.2 to about 10 mg/ml, from about 0.5 to about 8 mg/ml, from about 1 to about 5 mg/ml, from about 1.25 to about 3 mg/ml, from about 1.5 to about 2 mg/ml, or about 1.8 mg/ml methionine. In some embodiments, the radical scavenger moiety comprises a water-soluble free radical scavenger (e.g., antioxidant), ascorbic acid or its derivatives (e.g., derivatives such as thiols, sulfites, metabisulfites, bisulfites, etc.), or phosphonates/phosphonic acids, etc. In embodiments comprising methionine, the methionine can comprise any stereoisomer or combination of stereoisomers of methionine, as well as peptides comprising one or more methionine residues. In embodiments comprising peptides or other molecules having more than one methionine residue, each methionine residue is typically counted separately in determining the methionine concentration. Thus, in embodiments having polypeptides comprising two methionine residues, the methionine concentration, for the same number of peptides, would be double that of those embodiments comprising polypeptides having just one methionine residue each.

In particular methods, the hydrogel solutions comprise a metal ion chelator and a radical scavenging moiety. For example, in particular methods, the hydrogel solutions comprise 400 ppm chelator (e.g., EDTA) and 1.8 mg/ml scavenging moiety (e.g., methionine).

In certain embodiments, the methods herein produce hydrogels that after autoclaving and/or storage (e.g., storage for at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 25 weeks, at least 50 weeks, at least 1 year, at least 1.25 years, at least 1.5 years, at least 1.75 years, at least 2 years, at least 2.25 years, at least 2.5 years, at least 2.75 years, at least 3 years, at least 3.25 years, at least 3.5 years, or at least 3.75 years or longer, at temperatures such as 5° C., at typical ambient room temperature such as 20-25° C., at typical human body temperature (either internal or body surface), or at 40° C., comprise a greater viscosity than similar hydrogels (e.g., hydrogels made of the same or substantially similar types of polymers) that do not comprise metal ion chelators and/or radical scavenger moieties such as methionine. Thus, in other words, the methods herein create hydrogels that maintain their viscosity to a greater extent than hydrogels that are not created through the methods of the invention. The hydrogels produced through the methods of the invention can maintain at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 100%, at least 120%, at least 140%, at least 160%, at least 180%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at least 675%, at least 700%, at least 725%, at least 750%, at least 775%, at least 800%, at least 825%, at least 850%, at least 875%, at least 900%, at least 925%, at least 950%, at least 975%, or at least 1000% or more, greater viscosity over such periods (or after such periods) as compared to similar hydrogels that do not comprise a metal ion chelator and/or a radical scavenger moiety. While the hydrogels created through the methods of the invention maintain their viscosity, hydrogels that are not created by the methods of the invention will tend to lose their viscosity over such periods, thus leading to the hydrogels of the invention having a “greater” viscosity than the other hydrogels.

Also, in the various embodiments of the invention, the methods produce hydrogels that can comprise pharmaceutically acceptable hydrogels and can also optionally comprise one or more active agents (e.g., components that produce or help to produce a desired result or outcome, e.g., in a subject, such as a prophylactic and/or therapeutic result). Such active agents can include, e.g., vascular endothelial growth factor, lidocaine, local anesthetics, etc. The methods can produce hydrogels that can also optionally include, e.g., pharmaceutically acceptable excipients, buffers, etc. In some embodiments, the methods produce hydrogels suitable for topical wound dressings. In particular embodiments, the methods produce hydrogels comprising 400 ppm chelator (e.g., EDTA or DTPA) and/or 1.8 mg/ml scavenging moiety (e.g., methionine). In some embodiments, the methods can produce hydrogels that comprise 1.8 mg/mL methionine. In embodiments wherein the hydrogel is autoclave treated, any metal ion chelator can be added prior to such treatment, while any scavenging moiety (e.g., methionine) can be added after such treatment. In particular embodiments, the methods can produce hydrogels that comprise a composition as set forth in Table 1 herein. For example, the methods can produce a hydrogel that comprises (as measured in a one liter volume) 0.2, 0.6, or 1.8 g/L rh VEGF, 0.26 g/L succinic acid (or between about 0.2 and about 0.3 g/L), 0.76 g/L succinic acid, disodium salt, hexahydrate (or between about 0.7 and about 0.8 g/L), 37.8 g/L α,α-Trehalose, dihydrate (or between about 37 and 38 g/L), 0.036 g/L Polysorbate 20 (or between about 0.03 and 0.04 g/L), 30 g/L Hypromellose (hydroxypropyl methylcellulose) (or between about 25 and 35 g/L), 1.8 g/L Methionine-L (or between about 1.5 and 2.0 g/L), 0.1 g/L Benzalkoniun chloride solution (or between about 0.007 and 0.15 g/L), and water to qs to 1 L at a pH of about 4.7 to about 5.3, at a pH of about 5.0, or at a pH of 5.0. Such hydrogels can optionally be autoclave treated, wherein any chelator is optionally added prior to autoclaving and any scavenging moiety (or the rh VEGF) is added after autoclaving.

In some aspects, the invention comprises a hydrogel composition (optionally a pharmaceutically acceptable hydrogel) comprising a hydrogel solution (e.g., a solution/slurry comprising a hydrogel polymer and, e.g., buffer), one or more metal ion chelators (e.g., an autoclave stable chelator, such as an aminopolycarboxylate, EDTA, NTA, EDDS, EGTA, PDTA, or DTPA), and/or one or more radical scavenging moieties (e.g., methionine, a methionine derivative, or one or more peptides comprising methionine or a methionine derivative, etc.).

In various embodiments, the hydrogel is an autoclaved hydrogel, while in other embodiments, the hydrogel is a filtered or similarly treated hydrogel. In hydrogels that are autoclaved, the metal ion chelator, if present, can have been added prior to autoclaving, while the radical scavenger moiety, if present, can have been added after autoclaving.

In various embodiments, the hydrogels of the invention can comprise a plurality of polymer molecules, such as, but not limited to: polysaccharide molecules, cellulose molecules, cellulose derivative molecules, methylcellulose molecules, hydroxypropyl methylcellulose (hypromellose) molecules, carboxymethyl cellulose molecules, hydroxypropyl cellulose molecules, hydroxyethyl cellulose molecules, hyaluronic acid molecules, Carbopol® molecules (Noveon, Cleveland, Ohio) and their derivatives, alginate, sodium hyaluronate, gellan, carrageenan, pectin, gelatin, polyvinyl pyrrolidone, poloxamer, dextran, etc. The concentration of hydrogels herein can be quantified by percent polymer molecule to hydrogel solution (weight/volume). For cellulose/cellulose derivative hydrogels, the polymer molecule (e.g., methylcellulose, hydroxypropyl methylcellulose, hydroxyethylcellulose, Methocel® A4M, grade HEC hydroxyethyl cellulose, or Methocel® E4M) can comprise from about 1% to about 6% polymer molecule to hydrogel solution, from about 1.25% to about 5.5% polymer molecule to hydrogel solution, from about 1.5% to about 5.0% polymer molecule to hydrogel solution, from about 1.75% to about 4.5% polymer molecule to hydrogel solution, from about from about 2% to about 4% polymer molecule to hydrogel solution, from about 2.25% to about 4% polymer molecule to hydrogel solution, from about 2.5% to about 3.5% polymer molecule to hydrogel solution, or about 3% polymer molecule to hydrogel solution. It will be understood, however, that specific recitation of polymer concentrations in the various hydrogels herein should not necessarily be taken as limiting and that the methods (and the compositions) herein can also comprise lower or higher concentrations of hydrogel polymers in the hydrogels (e.g., depending upon the desired viscosity and upon the specific polymer used, etc.). For hydrogels based on non-cellulose/non-cellulose derivative polymers, the polymer concentration can be lower or higher than the polymer concentration for cellulose/cellulose derivative hydrogels. For example, hydrogels based on poloxamer can comprise, e.g., from about 15% to about 35%, from about 20% to about 30%, or about 25% polymer molecule to hydrogel solution, while pectin based hydrogels can comprise, e.g., about 3% to about 6%, or about 5% polymer to hydrogel solution. Furthermore, hydrogels based on polyvinyl pyrrolidone can comprise, e.g., about 10% polymer per hydrogel solution and hydrogels based on dextran can comprise, e.g., from about 5% to about 10% polymer per hydrogel solution. Those of skill in the art will be familiar with a range of appropriate polymer concentrations for numerous different hydrogel constructions.

In various embodiments, the hydrogels of the invention can comprise from about 50 to about 2000 ppm metal ion chelator (e.g., an aminopolycarboxylate, EDTA, NTA, EDDS, EGTA, PDTA, or DTPA), from about 100 to about 1500 ppm, from about 200 to about 1000 ppm chelator, from about 300 to about 500 ppm chelator, or about 400 ppm chelator. Alternatively or additionally, the hydrogels of the invention can comprise from about 0.01 to about 10 mg/ml, from about 0.05 to about 10 mg/ml, from about 0.1 to about 10 mg/ml, from about 0.15 to about 10 mg/ml, from about 0.2 to about 10 mg/ml, from about 0.5 to about 8 mg/ml, from about 1 to about 5 mg/ml, from about 1.25 to about 3 mg/ml, from about 1.5 to about 2 mg/ml, or about 1.8 mg/ml radical scavenging moiety (e.g., methionine) per volume of hydrogel. The radical scavenging moieties in the methods of the invention can comprise, but are not limited to, methionine or methionine derivatives, or short peptides comprising one or more methionine residues or methionine derivatives, etc. In some embodiments, the radical scavenger moiety comprises a water-soluble free radical scavenger (e.g., antioxidant), ascorbic acid or its derivatives (e.g., derivatives such as thiols, sulfites, metabisulfites, bisulfites, etc.), or phosphonates/phosphonic acids, etc. In embodiments comprising methionine, the methionine can comprise any stereoisomer or combination of stereoisomers of methionine, as well as peptides comprising one or more methionine residues. In embodiments comprising peptides or other molecules having more than one methionine residue, each methionine residue is typically counted separately in determining the methionine concentration. Thus, in embodiments having polypeptides comprising two methionine residues, the methionine concentration (for the same number of peptides) would be double that of those embodiments comprising polypeptides having just one methionine residue each. In particular embodiments, the compositions comprise 400 ppm chelator (e.g., EDTA or DTPA) and/or 1.8 mg/ml scavenging moiety (e.g., methionine). In some embodiments (either autoclaved or not), the compositions comprise 1.8 mg/mL methionine (optionally without chelator). In embodiments wherein the hydrogel is autoclave treated, the metal ion chelator can be added prior to such treatment, while any scavenging moiety (e.g., methionine) can be added after such treatment.

In particular embodiments, the hydrogel formulations herein can comprise a composition as set forth in Table 1 herein. For example, the composition can comprise (as measured in a one liter volume) 0.2, 0.6, or 1.8 g/L rh VEGF, 0.26 g/L succinic acid (or between about 0.2 and about 0.3 g/L), 0.76 g/L succinic acid, disodium salt, hexahydrate (or between about 0.7 and about 0.8 g/L), 37.8 g/L α,α-Trehalose, dihydrate (or between about 37 and 38 g/L), 0.036 g/L Polysorbate 20 (or between about 0.03 and 0.04 g/L), 30 g/L Hypromellose (hydroxypropyl methylcellulose) (or between about 25 and 35 g/L), 1.8 g/L Methionine-L (or between about 1.5 and 2.0 g/L), 0.1 g/L Benzalkoniun chloride solution (or 0.01% or 0.008% benzalkoniun chloride or between about 0.007 and 0.15 g/L), and water to qs to 1 L at a pH of about 4.7 to about 5.3, at a pH of about 5.0, or at a pH of 5.0. Such hydrogels can optionally be autoclave treated, wherein any chelator is optionally added prior to autoclaving and any scavenging moiety (or the rh VEGF) is added after autoclaving

In the compositions of the invention after autoclaving and/or storage (e.g., storage for at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 25 weeks, at least 50 weeks, at least 1 year, at least 1.25 years, at least 1.5 years, at least 1.75 years, at least 2 years, at least 2.25 years, at least 2.5 years, at least 2.75 years, at least 3 years, at least 3.25 years, at least 3.5 years, or at least 3.75 years or longer at temperatures such as 5° C., at typical ambient room temperature such as 20-25° C., at typical human body temperature (either internal or body surface), or at 40° C., the hydrogels of the invention comprise a greater viscosity than similar hydrogels (e.g., hydrogels made of the same or substantially similar types of polymers) that do not comprise the metal ion chelator and/or the radical scavenger moiety. Thus, in other words, the hydrogels of the invention maintain their viscosity to a greater extent than hydrogels that are not of the invention. The hydrogels of the invention can maintain at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 100%, at least 120%, at least 140%, at least 160%, at least 180%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at least 675%, at least 700%, at least 725%, at least 750%, at least 775%, at least 800%, at least 825%, at least 850%, at least 875%, at least 900%, at least 925%, at least 950%, at least 975%, or at least 1000% or more, greater viscosity over such periods (or after such periods) as compared to similar hydrogels that are not of the invention (e.g., those that do not comprise a metal ion chelator and/or a radical scavenger moiety). While the hydrogels of the invention maintain their viscosity, the hydrogels that are not of the invention will tend to lose their viscosity over such periods, thus leading to the hydrogels of the invention having a “greater” viscosity than the other hydrogels.

In the various embodiments, the hydrogels herein can comprise pharmaceutically acceptable hydrogels and/or can comprise one or more pharmaceutically active agent such as vascular endothelial growth factor, lidocaine, local anesthetics, etc. and/or pharmaceutically acceptable excipients, buffers, etc. In some embodiments, the hydrogels are topical wound dressings.

These and other features of the invention will become more fully apparent when the following detailed description is read in conjunction with the accompanying figures and claims.

### BRIEF DESCRIPTION OF THE DRAWINGS

FIG. 1, illustrates loss of gel viscosity over time in different lots of HPMC (without added chelator or radical scavenger) after autoclave sterilization.

FIG. 2, Panels A and B, illustrates loss of gel viscosity over time in HPMC (without added chelator or radical scavenger) in the presence of various concentrations of hydrogen peroxide at 5° C. (Panel A) and 40° C. (Panel B).

FIG. 3, Panels A and B, illustrates loss of gel viscosity over time in MC (without added chelator or radical scavenger) in the presence of various concentrations of hydrogen peroxide at 5° C. (Panel A) and 40° C. (Panel B).

FIG. 4, shows the percent of gel viscosity remaining for autoclaved HPMC and MC hydrogels comprising EDTA, in the presence of various concentrations of hydrogen peroxide.

FIG. 5, Panels A through C, illustrates the percent of gel viscosity remaining over time in different autoclaved HPMC gels in the presence of various concentrations of hydrogen peroxide at 40° C., with Panel A showing HPMC gels comprising 400 ppm EDTA, Panel B showing HPMC gels comprising 1.8 mg/ml methionine, and Panel C showing HPMC gels comprising 400 ppm EDTA and 1.8 mg/ml methionine.

FIG. 6, illustrates the mechanism of hydroxyl radical cleavage of 1,4-anhydrocellobitol.

FIG. 7, Panels A through C, illustrates the percent of gel viscosity remaining over time in different autoclaved HPMC gels in the presence of various concentrations of hydrogen peroxide at 5° C., with Panel A showing HPMC gels comprising 400 ppm EDTA, Panel B showing HPMC gels comprising 1.8 mg/ml methionine, and Panel C showing HPMC gels comprising 400 ppm EDTA and 1.8 mg/ml methionine.

FIG. 8, Panels A and B, illustrates comparison of release characteristics of methylcellulose and HPMC (hypromellose) hydrogel formulations with 0.2 mg/mL rh VEGF (Panel A) and 1.8 mg/mL rh VEGF (Panel B).

FIG. 9, Panels A through D, shows the percent viscosity, in various concentrations of H₂O₂ at 40° C. and 5° C., of a 3% HPMC hydrogel comprising methionine (Panels A and B) as well as the percent of oxidized VEGF in the presence of methionine in HPMC hydrogels (Panels C and D).

### DETAILED DESCRIPTION

The present invention comprises viscosity stable hydrogel compositions and methods to decrease or prevent viscosity loss in hydrogels. Hydrogels as a class are useful in a wide range of applications. Pharmaceutical/biomedical uses for hydrogels are especially useful. Particular formulations and constructions of hydrogels can be used in, e.g., contact lenses, lubrication of catheters, and as artificial skin or as artificial membranes (e.g., kidney membranes). Recent work has focused on the use of hydrogels as wound dressings or covers, e.g., in order to protect wounds from infection, to create a proper healing milieu, or to allow for drug delivery from the hydrogel itself. See, e.g., U.S. Ser. No. 11/455,017, U.S. Ser. No. 60/691,909, and U.S. Ser. No. 60/794,008.

However, it is often useful to have hydrogels of the proper viscosity in such applications. For example, in wound dressings, hydrogels with too low a viscosity may not properly adhere to wound areas. Furthermore, even if a hydrogel has the proper viscosity when manufactured or applied, environmental conditions can often decrease the viscosity, thereby making the hydrogel less useful. Such viscosity loss can arise in several situations. For example, autoclaving a hydrogel can decrease its viscosity. Of course, it will be appreciated that such problem is especially of concern for certain hydrogel pharmaceuticals. Hydrogel pharmaceuticals need to be pharmaceutically acceptable for medical uses. Particular medical uses (e.g., ophthalmic use, use in management of open wounds, etc.) typically require that the hydrogel be sterile or have a reduced bioburden as compared to compositions not used in such applications. However, convenient and inexpensive autoclave treatment can lead to viscosity loss. Additionally, in order to be transportable to where or when they are needed, it is desirable that pharmaceutical compositions be storage stable. Here too, however, problems can arise with traditional hydrogel compositions and methods of producing hydrogels because storage can cause or permit viscosity loss from free radicals, oxidation, etc. Such free radical damage can lead to decreases in viscosity which can render the hydrogel unsuitable for its intended pharmaceutical use.

The invention herein addresses the problems of viscosity loss (e.g., often due to autoclaving and storage, but also arising from other causes), especially for pharmaceutical compositions comprising cellulose hydrogels. It will be appreciated that while the methods and compositions are predominantly discussed herein in terms of pharmaceutical or medical applications, they are also capable of use with other systems and applications where viscosity loss is of concern. Also, even though in certain embodiments the invention is directed towards particular configurations and/or combinations of such aspects, those of skill in the art will appreciate that not all embodiments necessarily comprise all aspects or particular configurations (unless specifically stated to do so).

In brief, the current invention comprises methods of decreasing or preventing viscosity loss in hydrogels by producing hydrogels comprising a metal ion chelator (such as EDTA, EGTA, or DTPA) and/or a scavenger of free radicals (such as methionine). The invention also comprises hydrogel compositions comprising a metal ion chelator such as EDTA, EGTA, or DTPA (optionally added before autoclaving if the hydrogels are autoclaved) and/or a radical scavenging moiety such as methionine (optionally added after autoclaving if the hydrogel is autoclaved) which hydrogels show decreased viscosity loss as compared to similar hydrogels which do not comprise the chelator and/or scavenger moiety.

Thus, in various embodiments, the invention comprises viscosity stabilized hydrogels (and methods to create such hydrogels) including hydrogels that are autoclaved or autoclavable as well as hydrogels that are not autoclaved or autoclavable (e.g., hydrogels that are, e.g., filtered or otherwise treated to become sterile or to have a reduced bioburden or hydrogels that are not sterile or that do not have a reduced bioburden). For the hydrogels (and methods of their creation) that are autoclaved, the hydrogels can comprise a chelator (e.g., EDTA, etc.) and/or a radical scavenging moiety (e.g., methionine). Thus, some embodiments comprise a chelator but not a scavenging moiety, while others comprise a scavenging moiety but not a chelator. In other embodiments, the hydrogels comprise both a chelator and a scavenging moiety. In some embodiments, the chelator is added to the hydrogel prior to autoclaving while the scavenging moiety is added to the hydrogel after autoclaving.

Similarly, for the hydrogels and methods of their creation that are not autoclaved, the hydrogels can comprise a chelator (e.g., EDTA, etc.) and/or a radical scavenging moiety (e.g., methionine). Thus, some embodiments comprise a chelator but not a scavenging moiety, while others comprise a scavenging moiety but not a chelator. In other embodiments, the hydrogels comprise both a chelator and a scavenging moiety.

In particular embodiments, the hydrogels (and methods of their construction) comprise hydrogels comprising 400 ppm chelator (e.g., EDTA, etc.) and/or 1.8 mg/mL scavenging moiety (e.g., methionine). In some embodiments, such hydrogels are autoclaved, with the chelator being added prior to autoclaving and the scavenging moiety being added after autoclaving. In other such embodiments, the hydrogels are not autoclaved.

In other particular embodiments, the hydrogels (and methods of their construction) comprise 1.8 mg/mL scavenging moiety (e.g., methionine) and optionally no chelator. In some such embodiments, the hydrogel is not autoclaved, while in other such embodiments, the hydrogel is autoclaved prior to the scavenging moiety being added.

In other particular embodiments, the hydrogels (and methods of their construction) comprise 0.2, 0.6, or 1.8 mg/mL rh VEGF; 0.26 mg/mL succinic acid; 0.76 mg/mL succinic acid, disodium salt, hexahydrate; 37.8 mg/mL α,α-Trehalose, dihydrate; 0.036 mg/mL Polysorbate 20; 30 mg/mL hypromellose (hydroxypropyl methylcellulose); 1.8 mg/mL Methionine-L; 0.1 mg/mL Benzalkoniun chloride solution (or 0.01%, or 0.008%); and water to qs to 1.0 mL. In such embodiments, if the hydrogel is autoclaved, the methionine (and optionally the rh VEGF) is added after such. The pH of such embodiments can comprise from about pH 4.7 to about pH 5.3, about pH 5.0, or pH 5.0.

### DEFINITIONS

Before describing the present invention in detail, it is to be understood that the invention herein is not necessarily limited to use with particular hydrogels/hydrogel concentrations, or autoclave or storage systems or the like, which can, of course, vary. It is also to be understood that the terminology used herein is for the purpose of describing particular embodiments only, and is not necessarily intended to be limiting. As used in this specification and the appended claims, the singular forms “a,” “an,” and “the” include plural referents unless the context clearly dictates otherwise. Thus, for example, reference to “a chelator” optionally includes a combination of two or more chelators, and the like.

A compound or molecule described herein as “autoclave stable,” “temperature stable,” or the like, should be understood to be one that can be subjected to typical autoclave conditions of temperature and pressure and still be biologically and/or chemically active in its intended manner. Typical autoclave conditions (e.g., of temperature and pressure) will be quite familiar to those of skill in the art especially in regard to creating sterile or reduced bioburden compositions.

As an example, a metal ion chelator that is autoclave stable can be subjected to typical autoclave conditions and still be capable of chelating metal ions afterwards in a substantially similar fashion as it could before autoclaving. Alternatively, in some embodiments, a compound that is autoclave stable acts in a different (but intended) fashion than it did before autoclaving.

“Pharmaceutically acceptable” hydrogels herein are ones that are suitable for pharmaceutical use in a subject, including an animal or human. Particular pharmaceutically acceptable hydrogels that are applied to an open wound or to a mucous membrane (such as an eye area) are generally sterile (or have a reduced bioburden such that they are suitable for use in such applications). Pharmaceutical hydrogels are typically non-lethal and/or non-toxic in their intended use. Pharmaceutically acceptable hydrogels can optionally comprise an effective amount of one or more active agents/medicaments (e.g., VEGF, FGF, lidocaine). Additionally, preservatives and additives are optionally added to the hydrogel compositions to help ensure stability and sterility. For example, antibiotics and other bacteriocides, and the like are all optionally present in various embodiments of the invention herein. See below. A “pharmaceutical method” or “pharmaceutically acceptable method” such as a pharmaceutical method to decrease/stabilize hydrogel viscosity can be understood to produce products (e.g., hydrogels) that are pharmaceutically acceptable.

The term “effective amount” means a dosage or amount sufficient to produce a desired result. The desired result may comprise an objective or subjective improvement (e.g., long-term survival, decrease in number and/or size of wounds, effective prevention of infection, etc.) in the recipient receiving the hydrogel and any active agents in the hydrogel composition.

### Hydrogels and Hydrogel Construction

Hydrogels, in brief, are colloidal gels dispersed in water as a carrier medium. As stated previously, hydrogels find application in diverse fields and general techniques and methods in their basic construction are well known to those of skill in the art. However, hydrogel usage is limited in some areas, such as in some pharmaceutical applications, by their tendency to lose viscosity due to heat (e.g., from autoclaving or from contact with the body temperature of a subject) and/or storage (e.g., from polymer chain cleavage from reactions of free radicals).

This invention presents novel methods of decreasing or preventing viscosity loss in hydrogels by adding in one or more metal ion chelator prior to autoclaving of a hydrogel solution and/or by adding in one or more free radical scavenger prior to storage/use of the hydrogel (but typically after autoclaving). The invention also encompasses hydrogels and hydrogel compositions comprising such metal ion chelators and/or free radical scavengers.

In certain embodiments, herein, the hydrogels being increased in viscosity stability (i.e., those with decreased viscosity loss) and the methods used to produce them comprise cellulose based polymers (e.g., methylcellulose, etc.) to which a metal ion chelator such as EDTA is added prior to autoclaving and/or a radical scavenging moiety (e.g., methionine) is added after autoclaving. However, as will be appreciated, such description should not necessarily be taken as limiting. Thus, as stated above, the invention also includes hydrogels comprising a metal ion chelator and/or scavenging moiety in which the hydrogel has been filter sterilized rather than autoclaved, stabilized hydrogels that comprise a metal ion chelator but not a scavenging moiety, and stabilized hydrogels that comprise a scavenging moiety such as methionine but not a metal ion chelator. Also, as explained further below, alternative and/or additional components of the hydrogels (e.g., active agents) are also included within the scope of the invention.

In a similar fashion, while the hydrogels herein are often described as those for pharmaceutical or medical use such as for wound dressings (see below), it should be understood that the methods and compositions herein are also applicable to other fields (e.g., biotechnology research, etc.) that require hydrogels (e.g., autoclaved and/or stored hydrogels) which show decreased viscosity loss. Those of skill in the art will be familiar with additional applications for hydrogels (e.g., sterile or reduced bioburden hydrogels) having decreased viscosity loss.

In pharmaceutical preparations, cellulose derivatives are widely used to impart viscosity in topical, ophthalmic and vaginal formulations. As stated above, the viscosity of such hydrogels can be quite important in various applications. Therefore, the stability of the viscosity of these cellulose based hydrogel at different pH and temperature has been studied. See, Huikari, Pharmaceutica Fennicam 1986, 95(1):9-17; Huikari, et al., Acta Pharmaceutica Fennica 1989, 98: 231-238; and Ferdous, Pakistan J. Pharm. Sci. 1992, 5(2): 115-119. In addition to acid hydrolysis and high temperature, oxidative degradation has been thought to cause the loss of viscosity of the gel. See, Ha, et al., Phar. Res. 1999, S 1(4): 2867. For example, viscosity loss in HEC (hydroxyethyl cellulose) thickened latex paints has been attributed to chemical oxidants by measuring the oxidation reduction potential test. See, e.g., Vaughn, et al., Journal of Coatings Technology, Vol 52, No 660, January 1980, p71-74. Additionally, it has been reported that the thermal degradation temperatures and activation energies for the cellulose ethers such as methyl cellulose (MC), ethyl cellulose (EC), sodium carboxymethylcellulose (CMC), hydroxyethylcellulose (HEC) and hydroxypropyl methylcellulose (HPMC) were usually lower in air than in nitrogen. See, e.g., Li, et al., J. Applied Polymer Sci., 1999, 73: 2927-2936. During the sterilization process of some hydrogels, the gel can be exposed to high temperature which may cause the viscosity loss. Chu and Doyle (Pharm. Devel. Tech. 1999; 4(4): 553-559) have reported that significant viscosity loss in CMC gels that were to be used for compounding platelet derived growth factor occurred when the autoclave tank was flushed with oxygen, whereas viscosity was maintained when the tank was flushed with nitrogen.

Again, as stated previously, viscosity of various hydrogels can be adversely impacted by a number of factors. For example, peroxides, e.g., H₂O₂, t-butylhydroperoxide, commonly known as t-BHP, etc., can adversely impact hydrogel viscosity. It has been reported that, hydroxypropylcellulose (HPC) from different lots contained substantial amount of oxidant, equivalent to 50-890 nmole hydroperoxide per gram of HPC, with significant lot-to-lot and manufacturer-to-manufacturer variation. See, Wasylaschuk et al., J. Pharm. Sci. 2007, 96, 106-116. Because HPC is not water soluble, peroxide can be extracted into the aqueous medium without an interfering gel phase, and thus, peroxide level in HPC could be determined. However, the exact level of peroxide in methylcellulose or HPMC, cannot be determined by the same method because once the cellulose derivatives are placed in water, they are hydrated and become viscous liquids/gels which renders the assay inoperable. Since such cellulose derivatives are made in similar manner, one may expect that the peroxide level in HPC may be extended to MC or HPMC.

There has been further evidence of the presence of oxidant in a cellulose based preparation, which could affect protein stability in a hydrogel. Nguyen has reported that Relaxin was oxidized much faster in MC gel (Nguyen in Cleland J L, and Langer R, eds. Formulation and Delivery of proteins and peptides. ACS symposium series 567; 1994: 59-71) than in aqueous solution. Therefore the concern of oxidant in cellulose derivative is not limited to viscosity loss, but also possibly includes the degradation of any oxidation sensitive drug substance in the formulation.

Hydrogel Polymers

In general, hydrogels are composed of a plurality of water swollen crosslinked polymer molecules. Cross-linking may occur through, e.g., reaction between monomers, hydrogen bonds, van der Waals interactions, etc. Hydrogels can optionally comprise one type of polymer or a number of different types of polymers within the same hydrogel.

As will be appreciated by those of skill in the art, a wide range of different polymers can be used in construction of hydrogels in general and in construction of the hydrogels herein. For example, the hydrogels of the invention can comprise, polysaccharides, cellulose or cellulose derivatives, methylcellulose, ethyl hydroxyethyl cellulose, methyl ethyl hydroxyethyl cellulose, polyvinyl alcohols, polyethylene glycols, PNVP, PHEMA (and PHEMA derivatives), hydroxypropyl methylcellulose (HPMC or hypromellose), carboxymethyl cellulose, hydroxypropyl cellulose, hydroxyethyl cellulose, hyaluronic acid, Carbopol® (Noveon, Cleveland, Ohio) and its derivatives, alginate, sodium hyaluronate, gellan, carrageenan, pectin, gelatin, polyvinyl pyrrolidone, poloxamer, or dextran, etc. Additional hydrogel polymers that optionally can be used in the hydrogels of the invention can be found and obtained from common chemical/biological supply sources such as, but not limited to, Dow (Midlands, Mich.), Hercules (Wilmington, Del.), Sigma-Aldrich (St. Louis, Mo.), Akzo Nobel (Stenungsund, Sweden), etc. In particular embodiments, the hydrogels herein are comprised of molecules otherwise vulnerable to viscosity loss through interaction with free radicals, etc. when not used with the current invention.

Cellulose and cellulose derivatives are especially useful in a number of applications, especially for pharmaceutical use, due to their availability and medically innocuous nature. Cellulose derivates such as those listed above, e.g., methylcellulose, carboxymethyl cellulose, hydroxyethylcellulose, hydroxypropyl methylcellulose, etc., are optionally used in the invention. Again, it should be stressed that specific recitation of particular hydrogel polymers herein should not be taken as necessarily limiting and that other polymers are also encompassed within the invention. General construction of hydrogels by suspension of polymer molecules in an aqueous medium is well known to those of skill in the art and non-limiting examples of hydrogel construction are illustrated in the Examples section below.

While a basic hydrogel comprises a plurality of one or more polymer types, hydrogels in general and within the present invention can also comprise, e.g., crosslinkers, and other components, etc. Also as explained further below, hydrogels (including those of the invention) can also comprise one or more pharmaceutically active agent, such as a medicament (e.g., an antibiotic, VEGF or other growth factors, other proteins and peptides, other small molecules, etc.).

Hydrogels can comprise a wide range of concentrations. Again, while a number of hydrogel concentrations are described herein (see, e.g., Examples section), such recitation should not be taken as necessarily limiting. In some embodiments herein the hydrogels comprise a concentration of cellulose/cellulose derivative polymer molecule (e.g., methylcellulose, hydroxypropyl methylcellulose, hydroxyethylcellulose, methyl cellulose, hydroxyethyl cellulose, or hydroxypropyl methylcellulose) in the hydrogels that can comprise from about 1% to about 6% polymer molecule to hydrogel solution, from about 1.25% to about 5.5% polymer molecule to hydrogel solution, from about 1.5% to about 5.0% polymer molecule to hydrogel solution, from about 1.75% to about 4.5% polymer molecule to hydrogel solution, from about 2% to about 4% polymer molecule to hydrogel solution, from about 2.25% to about 4% polymer molecule to hydrogel solution, from about 2.5% to about 3.5% polymer molecule to hydrogel solution, or about 3% polymer molecule to hydrogel solution as measured by weight/volume. In some embodiments, the hydrogels herein comprise a viscosity of about 2000 to about 4000 cps. Again, it will be understood, however, that specific recitation of polymer concentrations in the various hydrogels herein should not necessarily be taken as limiting, that hydrogels comprising different polymers can comprise different polymer percentages, and that the hydrogels in the methods (and the compositions) herein can also optionally comprise lower or higher concentrations of polymers in the hydrogels. See above.

Metal Ion Chelators

In various embodiments of the invention, metal ion chelators are added to the hydrogels in order to help decrease viscosity loss (especially in regard to viscosity loss from autoclaving). It is thought that such chelators act to decrease viscosity loss by sequestering metal ions that would otherwise catalyze breakdown of the hydrogel through free radical reactions. However, the actual mode of action of chelators in decreasing viscosity loss should not necessarily be taken as limiting upon the invention. Additionally, in some embodiments to prevent reactions of free radicals, etc., the invention comprises autoclave stable antioxidants or other components that are not metal ion chelators, but which still act to protect gel viscosity from autoclave damage.

While the various descriptions of chelators herein focuses on EDTA, it will be appreciated that other metal ion chelators are also encompassed within the invention. Metal ion chelators are well known by those of skill in the art and include, but are not necessarily limited to aminopolycarboxylates, EDTA (ethylenediaminetetraacetic acid), EGTA (ethylene glycol-bis(beta-aminoethyl ether)-N,N,N′,N′-tetraacetic acid), NTA (nitrilotriacetic acid), EDDS (ethylene diamine disuccinate), PDTA (1,3-propylenediaminetetraacetic acid), DTPA (diethylenetriaminepentaacetic acid), ADA (beta-alaninediacetic acid), MGCA (methylglycinediacetic acid), etc. Additionally, some embodiments herein comprise phosphonates/phosphonic acid chelators.

In typical embodiments herein, the metal ion chelators are autoclave stable and are added to the hydrogel solution prior to the solution being autoclaved (if the solution is autoclaved). Of course, metal ion chelators can also be added to hydrogels that are not autoclaved. As described above, an autoclave stable chelator is one that does not lose (or does not totally lose) its chelating ability when autoclaved. Because the chelator is added prior to autoclaving, it can act to help prevent viscosity loss that would otherwise occur from the free radicals in the high heat and pressure of the autoclave. Thus, the hydrogels herein can be autoclave treated and therefore used as pharmaceutically acceptable hydrogels in applications requiring sterile or reduced bioburden hydrogels (e.g., those used on open wounds or in ophthalmic applications).

The hydrogels herein can comprise a range of chelator concentrations. Of course, recitation of specific concentrations should not be taken as necessarily limiting. In various embodiments, the hydrogels comprise chelator (e.g., EDTA or EGTA) concentrations from about 50 to about 2000 ppm chelator, from about 100 to about 1500 ppm, from about 200 to about 1000 ppm chelator, from about 300 to about 500 ppm chelator, or about 400 ppm chelator. In various embodiments, the concentration of chelator will vary depending upon, e.g., the specific chelator used.

Free Radical Scavenging Moieties

In various embodiments herein, free radical scavenging moieties are added to the hydrogel solutions herein in order to help decrease viscosity loss. Such moieties are thought to act by reacting with free radical components in the hydrogels (e.g., arising from peroxides such as H₂O₂ or BHP). Because the free radical components interact with the scavenging moieties instead of with the hydrogel polymers, the hydrogels are not degraded and thus do not lose viscosity. Of course, the actual mode of action of scavenging moieties in relation to hydrogel viscosity herein should not necessarily be taken as limiting upon the invention.

As with the description of chelating agents above, specific recitation of particular scavenging moieties herein should not necessarily be taken as limiting. Thus, while the description herein focuses on methionine, other scavenging moieties are also optionally used in various embodiments. In general, scavenging moieties herein will optionally act to “sacrifice” themselves. In other words, the scavenging moieties will optionally react with the free radicals (e.g., will be oxidized), rather than the hydrogel polymer, thus using up the free radical agent. Alternatively, or in addition to, methionine, embodiments can optionally comprise, e.g., water-soluble free radical scavengers, ascorbic acid or its derivatives (e.g., derivatives such as thiols, sulfites, metabisulfites, bisulfites, etc.), or phosphonates/phosphonic acids, etc. as scavenging moieties.

In embodiments comprising methionine, the methionine can comprise any stereoisomer or combination of stereoisomers of methionine, as well as peptides comprising one or more methionine or methionine derivative residue. Methionine derivatives can include, but are not limited to, e.g., N-Substituted methionine such as C₁-C₁₈ N-acyl derivatives of methionine, N-carbamoylmethionine (methionine urea), methionine hydantoin (cyclic urea), and N-carbethoxymethionine, various methionine sulfoxides, S-adenosyl methionine, etc. Those of skill in the art will be familiar with different methionine derivatives (especially those capable of use in pharmaceutical compositions). The peptides comprising such methionine or methionine derivative are optionally short peptides (e.g., between 2 and 5 amino acid residues in length), but can optionally comprise longer peptides such as 10, 20, 30 peptides or longer in length. Such peptides can optionally comprise any number of methionine or methionine residues (e.g., from one residue up to all residues in the peptide). In embodiments comprising peptides or other molecules having more than one methionine residue, each methionine residue is typically counted separately in determining methionine concentration. Thus, in embodiments having polypeptides comprising two methionine residues, the methionine concentration for the same number of polypeptides would be double that of an embodiment with polypeptides having just one methionine residue.

The scavenging moieties are typically added to the hydrogel solution after the hydrogel is autoclaved (if it is autoclaved) and, thus, do not necessarily have to be autoclave stable. However, autoclave stable scavenging moieties are also encompassed within the scope of the invention.

In the various embodiments herein comprising, e.g., methionine as the scavenging moiety, the hydrogel solutions can comprise from about 0.01 to about 10 mg/ml, from about 0.05 to about 10 mg/ml, from about 0.1 to about 10 mg/ml, from about 0.15 to about 10 mg/ml, from about 0.2 to about 10 mg/ml, from about 0.5 to about 8 mg/ml, from about 1 to about 5 mg/ml, from about 1.25 to about 3 mg/ml, from about 1.5 to about 2 mg/ml, or about 1.8 mg/ml scavenging moiety (e.g., methionine).

Hydrogel Viscosity Stabilization

The methods and compositions of the invention create/comprise hydrogels that have decreased viscosity loss when compared to methods/compositions that create/comprise hydrogels not having the aspects herein. Thus, in other words, the methods herein create hydrogels that maintain their viscosity to a greater extent than hydrogels that are not created through the methods of the invention. In some embodiments, the hydrogels and the hydrogels produced through the methods herein comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 100%, at least 120%, at least 140%, at least 160%, at least 180%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at least 675%, at least 700%, at least 725%, at least 750%, at least 775%, at least 800%, at least 825%, at least 850%, at least 875%, at least 900%, at least 925%, at least 950%, at least 975%, or at least 1000% or more, greater viscosity as compared to a similar hydrogel that does not comprise the metal ion chelator and/or scavenger moiety. While the hydrogels created through the methods of the invention maintain their viscosity, the hydrogels that are not created by the methods of the invention will tend to lose their viscosity over such periods, thus leading to the hydrogels of the invention having a “greater” viscosity than the other hydrogels.

Alternatively, the hydrogels and the hydrogels produced from the methods herein display less than 0.5%, less than 1%, less than 2%, less than 3%, less than 4%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, less than 99%, or less than 99.5% loss in viscosity from their original viscosity. The loss in viscosity (or decrease of loss in viscosity) can be measured over, or after, a period of storage time such as at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 25 weeks, at least 50 weeks, at least 1 year, at least 1.25 years, at least 1.5 years, at least 1.75 years, at least 2 years, at least 2.25 years, at least 2.5 years, at least 2.75 years, at least 3 years, at least 3.25 years, at least 3.5 years, or at least 3.75 years or longer.

The decrease in loss of viscosity can be seen over a wide range of temperatures. In some embodiments, the decrease in loss in viscosity is seen at 5° C., at typical ambient room temperature such as 20-25° C., at 40° C., at typical internal body temperature for a subject (e.g., human), at typical surface body temperature for a subject (e.g., human), etc.

The viscosity of the various hydrogels herein can optionally be measured through a number of different ways. Those of skill in the art will be familiar with various methods to determine viscosity (and changes in viscosity) in hydrogels. For example, hydrogel viscosity can optionally be measured through use of a rheometer such as those available from Anton Paar (Graz, Austria). Other devices and procedures to determine viscosity can be found at Paar's website (www (dot) anton-paar (dot) corn). It will be appreciated that the viscosity value of a particular hydrogel may be different depending on the procedure/device used to determine viscosity.

Uses of Hydrogels having Decreased Loss of Viscosity

As stated previously, hydrogels in general can be used for a number of purposes, including as lubricants, in ophthalmic applications, etc. The methods and compositions of the invention are especially suited to preparation of pharmaceutically acceptable hydrogels (e.g., for use as topical wound dressings).

In order to be used as pharmaceutical compositions on open wounds or with ophthalmic applications, it is often desired that hydrogels (such as those of the invention) be sterile or have reduced bioburden. Because various methods of the invention produce autoclave stable hydrogels, autoclaving is conveniently available as a way to produce the hydrogels. Those of skill in the art will be extremely familiar with autoclaving. Autoclave treatment of materials is widespread, especially in regard to various medical preparations. The specific autoclave conditions that are optionally used with the hydrogels and methods herein can vary from embodiment to embodiment. For example, hydrogels herein are optionally exposed to moist heat at 121° C. and 15 pounds pressure for 45 minutes. However, such conditions should not necessarily be taken as limiting. Other times, temperatures, and pressure values are also optionally utilized with the invention, depending upon, e.g., the specific compositions of the hydrogels involved, etc. Alternatively, in various embodiments herein, the hydrogels are not autoclaved, but rather are filtered or otherwise treated to produce a sterile or reduced bioburden hydrogel. For other particular embodiments (e.g., those not requiring sterility or reduced bioburden), the hydrogels are not autoclaved, filtered, etc.

As mentioned, hydrogels of various compositions can be used to promote or aid in wound healing. The hydrogels optionally aid in wound healing through a number of avenues. For example, hydrogels can help in creating and/or promoting a favorable healing environment/milieu on, or within, wounds by controlling humidity/moisture of the wound area, since moist environments can aid in fibroplasia re-epithelization of wounds. Additionally, hydrogels in some embodiments can absorb fluid discharge or exudates from a wound, thereby preventing excess buildup of such fluid. Hydrogels can also aid in debridement of wounds and wound areas through enhancing autolytic debridement. Further use of hydrogels in wound treatment is described in, e.g., Argen, Acta. Derm. Venerel., 1998, 78:119-122; Ishihara, et al., J. Artif. Organs, 2006, 9(1):8-16; Eisenbud, et al., 2003, Ostomy Wound Manage., 49(10):52-57; U.S. Pat. No. 5,457,093; U.S. Pat. No. 6,809,231; U.S. Pat. No. 6,706,279; U.S. Pat. No. 4,717,717; U.S. Pat. No. 6,238,691; U.S. Pat. No. 6,861,067; U.S. Pat. No. 6,258,995; and U.S. Pat. No. 7,083,806.

Hydrogels of the invention can be used on a number of different wound types, e.g., dry necrotic wounds such as pressure sores/diabetic wounds. Additionally, hydrogels herein can be used with exudating wounds (e.g., venous/arterial leg ulcers). The hydrogels can also be used with burns (e.g., to control fluid exudates, to provide endothermic cooling, and to aid in reduction in keloid scar formation), and as skin graft dressings and the like. The hydrogels can also be used with internal incisions and internal wounds such as gastric ulcers.

Additionally, the hydrogels of the invention can also be used with, or as a carrier for, suitable medicaments or active agents for wound treatment. For example, various embodiments herein can optionally comprise such additional agents as, e.g., antibiotics (e.g., aminoglycosides, etc.); anti-fungal agents; analgesics (e.g., novocaine, lidocaine and other related compounds); anti-inflammatory compounds (both steroidal and non-steroidal); growth factors (e.g., vascular endothelial growth factor, nerve growth factor, transforming growth factor-alpha, epidermal growth factor, platelet-derived growth factor, fibroblast growth factor, placental growth factor, connective tissue growth factor, keratinocyte growth factor, transforming growth factors etc.); debridement enzymes such as subtilysin or papain; vitamins and amino acids (e.g., to help diminish scarring), etc.

The hydrogels can provide a controlled delivery system for any such active agents (e.g., drugs/medicaments) at a wound site. Controlled delivery allows release sufficient to maintain a therapeutic level of the active agent over an extended period of time. Such increased contact time at the wound site can be necessary to achieve the desired goal (e.g., wound improvement).

It will be appreciated that such illustrations should not be taken as necessarily limiting on the invention and that additional and alternative active agents are also optionally used with the invention. Also it will be appreciated that the suitable concentrations/amounts of any active agent (e.g., drug/medicament) will be readily apparent to those skilled in the art. For example, concentrations/amounts of particular active agents can depend on their purpose, half-life, cost, etc.

The hydrogels herein can be applied directly to a wound site or can be used in conjunction with an appropriate substrate or scaffolding when applied. See, e.g., U.S. Pat. No. 6,406,712; U.S. Pat. No. 4,226,232; U.S. Pat. No. 6,399,091; and U.S. Pat. No. 4,452,892.

Active Agents and Hydrogel Compositions comprising Active Agents

In particular embodiments, the hydrogels and the methods herein comprise VEGF as an active agent, as well as optionally other constituents. In certain embodiments, the hydrogels herein and the hydrogels produced with the methods herein comprise, e.g., between 0.10 and 2.00 g/L; between 0.25 and 1.75 g/L, between 0.50 and 1.25 g/L, between 0.75 and 1.00 g/L, 0.2 g/L, 0.6 g/L, or 1.8 g/L VEGF as an active agent. In some such embodiments, the hydrogels comprise about 3% HPMC in succinate buffer at about pH 4.7 to about pH 5.3, at about pH 4.9 to about pH 5.2, about pH 5.0, or at pH 5.0. The VEGF can optionally be mixed with the hydrogel after autoclave treatment of the hydrogel (if it is autoclaved) and the subsequent hydrogel can be applied to an open wound, e.g., as indicated herein or as indicated in the references cited herein. Further information on VEGF and VEGF in hydrogels can be found in U.S. Ser. No. 11/455,017, U.S. Ser. No. 60/691,909, and U.S. Ser. No. 60/794,008, the specifications of which are incorporated herein in their entirety for all purposes.

The VEGF in various embodiments herein can comprise recombinant human vascular endothelial growth factor (rh VEGF, telbermin). VEGF has been investigated as a therapy for a number of indications including cardiovascular diseases, bone growth, and diabetic foot ulcers, etc. For treating a number of medical conditions, e.g., foot ulcers, the dosage form comprises a hydrogel such as those of the instant invention. Additional information on VEGF and its medical use is found in U.S. Ser. No. 11/455,017, U.S. Ser. No. 60/691,909, and U.S. Ser. No. 60/794,008. The term “VEGF” as used herein refers to vascular endothelial cell growth factor protein. Such VEGF can comprise the 165-amino acid human vascular endothelial cell growth factor, and related 121-, 145-, 183- 189-, and 206-, (and other isoforms) amino acid vascular endothelial cell growth factors, as described by Leung, et al., Science 246:1306 (1989), and Houck, et al., Mol. Endocrin. 5:1806 (1991) together with the naturally occurring allelic and processed forms of those growth factors. See also: Ferrara, et al., Endocr Rev 18:1-22 (1997); Henry and Abraham, Review of Preclinical and Clinical Results with Vascular Endothelial Growth Factors for Therapeutic Angiogenesis, Current Interventional Cardiology Reports, 2:228-241 (2000); and U.S. Pat. Nos. 5,332,671 and 6,899,882. VEGF can also optionally include VEGF variants, e.g., biologically active polypeptides having at least about 80% amino acid sequence identity to a corresponding VEGF native sequence polypeptide, or fragment thereof. Such variants include, for instance, polypeptides wherein one or more amino acid residues are added or deleted at the N- and/or C-terminus of the polypeptide or where one or more amino acids are changed from the native sequence. Ordinarily, a variant will have at least about 80% amino acid sequence identity, or at least about 90% amino acid sequence identity, or at least about 95% or more amino acid sequence identity with the native sequence polypeptide, or fragment thereof. Analogues or variants are defined as molecules in which the amino acid sequence, glycosylation, or other feature of native VEGF has been modified covalently or noncovalently. The invention also includes use of VEGF variants having VEGF activity and which act as agonists of the VEGF receptors, e.g., VEGFR1 and/or VEGFR2 agonists, in place of or in addition to VEGF. In some embodiments, VEGF₁₆₅ is added to the stabilized hydrogels of the invention.

The active agent, e.g., VEGF in the stabilized hydrogels herein can be optionally administered to one or more “subject.” For purposes of the invention a subject herein refers to any animal, e.g., a mammal, such as, but not limited to humans, domestic and farm animals, and zoo, sports, or pet animals, such as dogs, horses, cats, cows, sheep, pigs, etc.

In various embodiments comprising an active agent, such as VEGF, the active agent is typically present in a concentration or concentration range such that it comprises an effective amount. The term “effective amount” or “therapeutically effective amount” in such context refers to an amount of active agent effective to produce a desired result, e.g., to accelerate or improve wound healing in a subject or prevent recurrence of a wound in a subject. Administration an active agent “in combination with” one or more further agents includes simultaneous (concurrent) and/or consecutive administration of the active agent and the other agent(s) in any order. See below. “Treatment” refers to both therapeutic treatment and prophylactic or preventative measures. Those in need of treatment include those already with a disorder (such as an open would or such) as well as those in which a disorder is to be prevented. “Wound healing” refers a condition that would benefit from treatment with a hydrogel of the invention (e.g., a hydrogel of the invention comprising an active agent such as VEGF).

In addition to administration of an active agent by itself in a stabilized hydrogel, the following (as well as other) agents can also be combined with the hydrogel treatments: VEGF, platelet-derived growth factor (PDGF) such as Becaplermin (rhPDGF-BB), e.g., Regranex® from Johnson & Johnson (see, e.g., U.S. Pat. Nos. 5,457,093; 5,705,485; and, 5,427,778; Perry, et al., Cont. Clin. Trials 23:389-408 (2002)); adenosine-A2A receptor agonists (e.g., MRE0094 from King Pharmaceuticals); keratinocyte growth factor (e.g., KGF-2, repifermin from Human Genome Sciences); lactoferrin (LF), e.g., from Agennix, Inc.; thymosine beta-4 (e.g., TΓ4 from ReGeneRx Biopharmaceuticals); thrombin-derived activating receptor peptide (e.g., TP508; Chrysalin® from Chrysalis Biotechnology, Inc.); adenoviral vector encoding platelet-derived growth factor (PDGF-B) such as GAM501 from Selective Genetics; autologous bone marrow stem cells (BMSC) (see, e.g., Badiavas & Falanga, Arch Dermatol, 139:510-16 (2003); and, engineered living tissue grafts (e.g., Apligraf, etc.). Antibiotic and antiseptic ulcer agents can also be combined with administration of the active agent/hydrogel. Active agent hydrogel administration can also be administered along with immunosuppressive treatment (e.g., corticosteroids, radiation therapy, chemotherapy) or cancer treatment. Growth factors such as EGF, FGF, IGF, PDGF, and TGF can also be administered in coordination with stabilized hydrogels (e.g., comprising an active agent). See, e.g., Nagai, Exprt Opin Biol. Ther 2:211-18 (2002). Examples of such growth factors include platelet derived growth factor (PDGF-A, PDGF-B, PDGF-C, and PDGF-D), insulin-like growth factor I and II (IGF-I and IGF-II), acidic and basic fibroblast growth factor (aFGF and bFGF), alpha and beta transforming growth factor (TGF-α and TGF-β (e.g., TGF-beta 1, TGF beta 2, TGF beta 3)), epidermal growth factor (EGF), and others. See Id. Positive angiogenesis agents that can be combined with an active agent in the hydrogels include, but are not limited to, e.g., HGF, TNF-α, angiogenin, IL-8, etc. (see, e.g., Folkman, et al. J. Biol. Chem. 267:10931-10934 (1992); Klagsbrun, et al. Annu. Rev. Physiol. 53:217-239 (1991)), angiogenesis factors (such as Angiopoietins 1 and 2, Tie-2, Alpha-5 integrins, Matrix metalloproteinases, Nitric oxide (NO), COX-2, TGFbeta and receptors, VEGF and receptors), and other agents described herein or known by those of skill in the art, etc. In one embodiment, the active agent is VEGF, which, as described herein, can be combined with any other active agent.

In certain embodiments, the formulations to be used for in vivo administration are sterile or have reduced bioburden (e.g., those hydrogels to be applied to open wounds, etc.). This is readily accomplished by filtration through sterile filtration membranes for various components, and/or autoclave treatment of the hydrogel (prior to addition of components such as VEGF or methionine or other active agents or other components that would be harmed through autoclaving).

Typically for wound healing, active agents in stabilized hydrogels are formulated for site-specific delivery. When applied topically, the active agent stabilized hydrogel compositions also may be impregnated into sterile dressings, transdermal patches, plasters, and bandages. Oxidized regenerated cellulose/collagen matrices can also be used, e.g., Promogran™ Matrix Wound Dressing or Promogran Prisma Matrix™.

Sustained-release preparations can also be prepared with the stabilized hydrogels having active agents. Suitable examples of sustained-release preparations include semipermeable matrices of solid hydrophobic polymers containing active agents (e.g., VEGF), which matrices are in the form of shaped articles, e.g. films, or microcapsules. Of course, it will be appreciated that certain hydrogels release proteins and/or other components over different time periods than others, and particular choices can be based on such differences.

For obtaining a gel formulation, the active agent formulated in a liquid composition can be mixed with an effective amount of a water-soluble hydrogel polymer or the like to form a gel of the proper viscosity to be applied topically. In various embodiments of the invention, the gelling agent herein is one that is, e.g., inert to biological systems, nontoxic, simple to prepare, and/or not too runny or viscous, and will not destabilize the active agent held within it.

In certain embodiments of the invention, the hydrogel polymer is an etherified cellulose derivative, in another embodiment the polymer is one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose. In particular embodiments, the hydrogels comprise MC or HPMC polymers. See above.

In various embodiments, the active agent in stable hydrogel formulations (e.g., “single dose” formulations that are used up in one application or “multiple use” formulations that are to be used up over a period of time) of the invention can comprise methylcellulose USP/NF (MC, Methocel® A4M) or hypromellose USP/NF (HPMC; hydroxypropylmethyl cellulose, Methocel® E4M) respectively as the gelling agent and EDTA, EGTA, or DPTA, etc., and/or methionine as the hydrogel stabilizer. Methylcellulose and HPMC share the same cellulose backbone, but have different substitutions of certain hydroxyl (HO-) groups by methoxyl (CH30-), for MC, and hydroxypropyl (CH3CH(OH)CH20-) plus methoxyl groups, for HPMC.

Therapeutic formulations of active agents, e.g., VEGF, with or without additional therapeutic agents combined with the active agents, are formulated into stabilized hydrogels. An exemplary embodiment of such formulations is shown in Example 2 (see, e.g., Table 1). It will be appreciated that other exemplary embodiments having an active agent (e.g., such as VEGF) can also comprise a chelator (e.g., EDTA) as described herein and that, if the embodiment does comprise such, the chelator can optionally be added to the hydrogel prior to autoclaving if the hydrogel is autoclaved. Furthermore, other embodiments can optionally not be autoclaved, but rather, can be filtered or otherwise treated to be sterilized or have reduced bioburden.

Dosages and desired drug concentrations of pharmaceutical compositions of the invention may vary depending on the particular use envisioned. See U.S. Ser. No. 11/455,017, U.S. Ser. No. 60/691,909, and U.S. Ser. No. 60/794,008 for examples of wound treatment with stabilized VEGF hydrogel compositions and information and references on determining effective dosage in various applications.

The dosage to be employed is dependent upon various factors. In certain usages, depending on the type and severity of the condition of the subject, about 1 ug/kg to 50 mg/kg (e.g. 0.1-20 mg/kg) of an active agent (e.g., VEGF) and/or an additional agent, is a candidate dosage for administration to a subject, either through one or more separate administrations, or by continuous application. Guidance as to particular dosages and methods of delivery is provided in the literature. In one embodiment, the effective amount of VEGF administered in a stabilized hydrogel to a subject's surface is about 20 μg/cm² to about 250 μg/cm². The agent is suitably administered to the subject over a series of treatments or at one time.

For repeated administrations over several days or longer, depending on the condition, the treatment can be sustained until a desired suppression of disease symptoms occurs, e.g., complete closure of a wound, or reduction in wound area. However, other dosage regimens may be useful. Typically, the clinician will administer stabilized hydrogels of the invention comprising an active agent until a dosage(s) is reached that provides the required biological effect. The administration of the effective amount of an active agent can be daily or optionally a few times a week, e.g., at least twice a week, or at least three times a week, or at least four times a week, or at least five times a week, or at least six times a week. The progress of the therapy of the invention is easily monitored by conventional techniques and assays.

The therapeutic compositions of the invention are typically administered topically to the subject. In one embodiment of the invention, the hydrogel comprising the active agent (e.g., VEGF) is in a formulation of a topical gel, e.g., in a pre-filled syringe or container.

Kits and Articles of Manufacture

In some embodiments, the invention provides a kit or an article of manufacture containing materials useful for the methods and compositions described herein. Such kits can optionally comprise one or more containers, labels, and instructions, as well components for construction of viscosity stable hydrogels and/or actual viscosity stable hydrogels.

The kits can also optionally comprise one or more active agents (e.g., pharmaceutical components or medicaments such as VEGF, various antibiotics, various antifungal agents, etc.) comprised as part of the hydrogel, or to be comprised as part of the hydrogel. The kits can optionally include aluminum tubes or other containers (e.g., of glass, plastic, nylon, cotton, polyester, metal, etc.) to store the hydrogels or in which to mix/prepare the hydrogels as well as one or more devices with which to apply a hydrogel herein to a subject (e.g., a human in need of a wound dressing, etc.). In some embodiments, the device with which to apply the hydrogel to the subject comprises the container in which the hydrogel is stored and/or mixed/prepared.

The kits can also optionally include additional therapeutic components in addition to the hydrogels and/or hydrogel components of the invention, e.g., buffers, diluents, filters, dressings, bandages, applicators, gauze, barriers, semi-permeable barriers, tongue depressors, needles, and syringes, etc.

In many embodiments, the kits comprise instructions (e.g., typically written instructions) relating to the use of the kit to create a viscosity stable hydrogel and/or to use a viscosity stable hydrogel (e.g., as a wound dressing). In some embodiments, the kits comprise a URL address or phone number or the like for users to contact for instructions or further instructions. The kits can be unit doses, bulk packages (e.g., multi-dose packages), or sub-unit doses.

### EXAMPLES

The following are examples of general techniques and the like (e.g., for decreasing or preventing viscosity loss in hydrogels). It will be appreciated that such descriptions and examples are not necessarily limiting upon the methods, compositions, etc. of the invention, unless specifically stated to be so. It is understood that examples and embodiments described herein are for illustrative purposes only and that various modifications or changes in light thereof will be suggested to persons skilled in the art and are to be included within the spirit and purview of this application and within the scope of the appended claims.

### Example 1

To examine the effect of EDTA and methionine on stability (both during/soon after autoclaving and during storage) of hydrogel viscosity, colloidal gels were prepared from different powder types, namely, methylcellulose (MC) USP/NF, (e.g., Methocel® A4M grade (Dow, Midland, Mich.)), and hypromellose, also known as hydroxypropyl methylcellulose, (HPMC) USP/NF, (e.g., Methocel® E4M grade (Dow, Midland, Mich.). Various hydrogels comprising EDTA and/or methionine and hydrogels without EDTA and/or methionine were prepared as described.

Preparation of Hydrogel Solutions

Sodium succinate 6-hydrate and succinic acid were purchased from JT Baker (Phillipsburg, N.J.). Hydrogen peroxide (30% solution) was purchased from VWR International (West Chester, Pa.) and EDTA (disodium edetate) and methionine were purchased from Sigma Aldrich (St. Louis, Mo.).

In the comparison between hypromellose lots (see below), two hydrogels were prepared from each of four lots of hypromellose powder. 5 mM NaSuccinate pH 5.0 buffer was weighed into 500 mL Kimex bottles and heated to 70° C. Hypromellose powder was added as 4.7% weight/weight to the buffer. The exact amount of powder added was determined by weight to ensure that any variation was not due to differences in powder transfer. The powder was dispersed into the buffer to form a slurry by swirling the bottle. One bottle of each lot of gel to be tested was then autoclaved for 45 minutes at an exposure of 121° C. and 15 psi. After the autoclave cycle, the bottles were shaken to disperse the precipitated hypromellose and then weighed. Further buffer was added to make a 3% gel (based on powder weight) and the bottles were placed on a bottle roller at 2-8° C. until of homogenous appearance. Bottles that were not autoclaved had buffer added to the slurry to make a 3% gel and were then placed on a bottle roller at 2-8° C.

For determination of the effect of H₂O₂ and EDTA during autoclave heat treatment of hydrogels (see below), one stock gel for hypromellose and one for MC were prepared. 5 mM sodium Succinate pH 5.0 buffer was heated to 70° C. Gel powder was then added to make a 4.7% gel. The suspension was mixed with a Heidolph RZR 2021 propeller mixer until it formed a uniform hydrogel. This gel was then allowed to sit at room temperature until all bubbles had risen out of it. 10 g of the gel was then weighed into 100 mL Kimex bottles. Aliquots of EDTA and/or peroxide stock solutions were added to each gel to achieve desired final concentrations. The gels were autoclaved for 20 minutes exposure at 121° C. and 15 psi. Bottles were shaken after the autoclave cycle to disperse any precipitated MC or hypromellose and the bottles were placed on bottle rollers at 2-8° C.

For determination of the effect of H₂O₂ and EDTA on stored hydrogels (see below), one stock gel for hypromellose and one stock gel for MC were prepared for each study. 5 mM NaSuccinate pH 5.0 buffer was heated to 70° C. and the appropriate cellulose powder added to make a 4.7% gel. The suspensions were mixed with a Heidolph RZR 2021 propeller mixer until they formed uniform hydrogels and were then transferred to Kimex bottles. The gels were autoclaved for 45 minutes exposure at 121° C. and 15 psi. The bottles of hydrogels were shaken after the autoclave cycle to disperse any precipitated MC or hypromellose and put on bottle rollers at 2-8° C. Different formulations of each gel type were made by transferring and weighing approximately 13 g of 4.7% gel into 50 mL polypropylene tubes. Buffer alone, buffer with EDTA, buffer with methionine or buffer with EDTA and methionine were added for a final concentration of 3% gel, with 400 ppm EDTA and 1.8 mg/mL methionine in appropriate gels. To these gels 0.3% H₂O₂ was added to 0 ppm, 2 ppm, 5 ppm, 20 ppm or 50 ppm concentration. Gels were then mixed at 2-8° C. on a bottle roller. After mixing, 1 g of each gel was dispensed into 5 cc West glass vials with serum stoppers. These vials were stored at 5° C. and 40° C.

For safety reasons, all autoclaved bottles were fitted with caps having filtered vents.

Viscosity Measurement

Viscosity was determined using a Paar Physica UDS 200 cone and plate rheometer (Graz, Austria) at a constant shear rate of 120/s (about 20 RPM) with a 25 cm cone which was temperature controlled to 25° C. A positive displacement pipette was used to place approximately 200 ul of gel on the plate. Excess was removed once the cone was lowered into measuring position. Each reported viscosity value was the average from three measurements for each sample. In general without addition of H₂O₂ the viscosity of MC and hypromellose gels is about 2800 cps under the conditions measured.

Hydrogel Viscosity Loss Due to Autoclave Sterilization

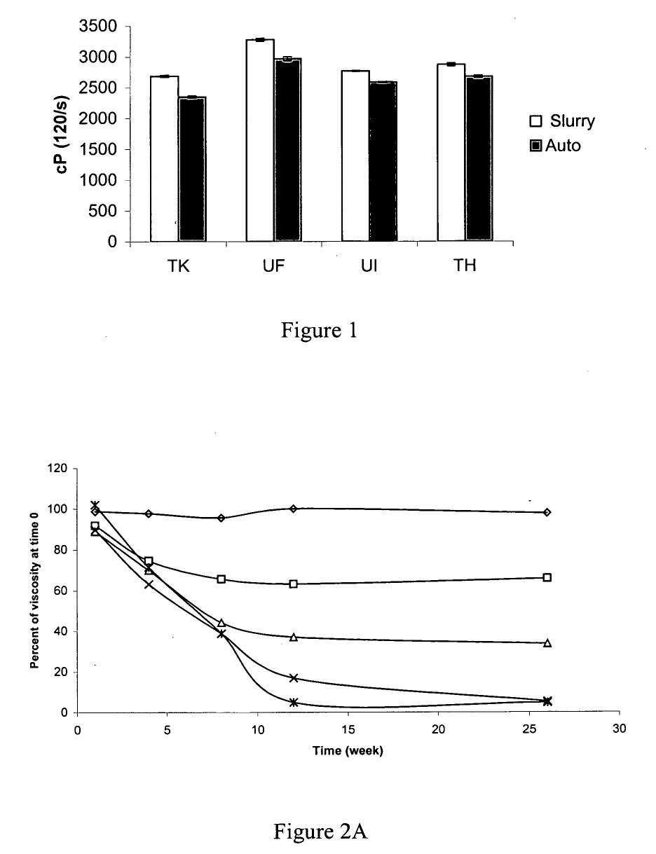

Viscosity loss due to the autoclave sterilization process has been seen with various hydrogels, e.g., sodium carboxymethylcellulose (see, e.g., Chu, et al., Pharm. Devel. Tech., 1999; 4(4): 553-559). To evaluate susceptibility of uncharged cellulose derivatives (which can experience fewer unwanted side effects with protein components) such as MC or hypromellose to autoclave sterilization, four lots of hypromellose raw material, named as lot TK, UF, UI and UH, were made into 3% gels and studied to compare the gel viscosity before and after autoclave sterilization. All four showed varying degrees of viscosity loss. See FIG. 1 which shows the results of 3% hypromellose gel viscosity both before (dot column indicating the un-autoclaved hydrogel slurry) and after (grid column indicating the post-autoclaved hydrogel) autoclave sterilization when each hydrogel was made from different lots of hypromellose powder. Lot numbers of the hypromellose are labeled as TK, UF, Ul and TH.

Effect of Hydrogen Peroxide Concentration and Temperature on Gel Viscosity

The examples herein track viscosity stabilization (“accelerated oxidative damage”) in hydrogels by monitoring the effects arising from various levels of hydrogen peroxide. Up to 20 or 100 ppm of hydrogen peroxide were used to simulate the “worst case” conditions of peroxide levels that induce oxidative reaction. These levels appear higher than the “worst case” levels for HPC in Wasylaschuk (above), e.g., 0.96 ppm peroxide when made into a 3% gel. The peroxide levels herein were also chosen based on Dahl (see above). The various examples examine the effect of a metal ion chelator (edetate disodium, ethylenediaminetetraacetate) on preventing viscosity loss during autoclave sterilization of gels (e.g., HPMC) in the presence of hydrogen peroxide and the effect of EDTA and a scavenging moiety (methionine), singularly or in combination, on viscosity loss over long term (6 months) storage in the presence of hydrogen peroxide.

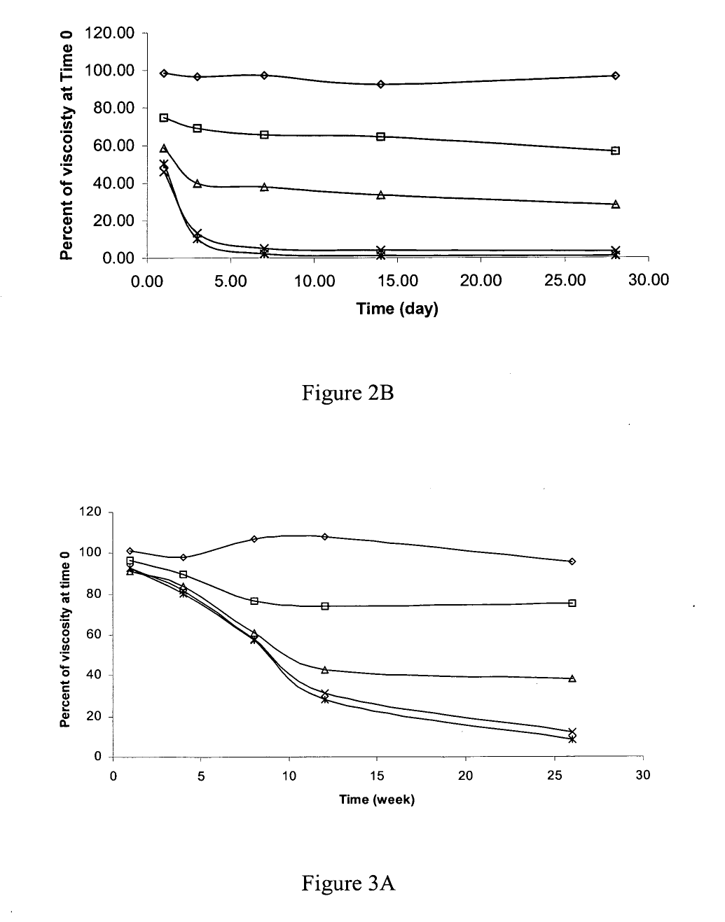

FIGS. 2A and 2B show that hydrogen peroxide, at least above 5 ppm, caused a rapid, and significant decrease in the viscosity of HPMC in gels not having EDTA or methionine. Thus, as can be seen, FIG. 2 shows the percent of the gel viscosity remaining as compared to time zero in the presence of various concentrations of H₂O₂ at 5° C. (Panel A) and 40° C. (Panel B) for 3% hypromellose (HPMC, METHOCEL® E4M) gel. In FIG. 2, ⋄=0 ppm H₂O₂, □=2 ppm H₂O₂, Δ=5 ppm H₂O₂, x=20 ppm H₂O₂, and *=50 ppm H₂O₂. Hydrogen peroxide was added to the hydrogels to simulate the peroxide that can arise in hydrogels. Peroxide can decrease viscosity since many hydrogels (e.g., cellulose hydrogels) comprise C—H bonds that are vulnerable to radical mediated oxidation which can lead to polymer cleavage and decrease in viscosity. However, as explained above, the protection shown against hydrogen peroxide oxidation is expected to be applicable to viscosity loss arising from other/similar free radical or oxidative actions (e.g., reactions arising not only from H₂O₂ but also from non-hydrogen peroxide origin, e.g., BHP). As can be seen from FIG. 2, the higher the concentration of hydrogen peroxide, the greater the loss of gel viscosity. For example, at 5° C., (FIG. 2A, note scale is in weeks) the percent of remaining viscosity at the 8th week was 70%, 45%, 40% and 40% for the gels containing 2 ppm, 5 ppm, 20 ppm and 50 ppm hydrogen peroxide, respectively.

At 40° C., (FIG. 2B, note the scale is in days) the percent of remaining viscosity at the 3^{rd} day compared to that at time zero was 70%, 40%, 15% and 10% for the gels containing 2 ppm, 5 ppm, 20 ppm and 50 ppm hydrogen peroxide, respectively. The decrease of viscosity reached a plateau after about 3 days at 40° C., while the viscosity of the gels at 5° C. with 20 ppm and 50 ppm H₂O₂ still continued to decrease. The viscosity loss of the gels with 2 ppm and 5 ppm H₂O₂ at 5° C. reached their plateau after about 12 weeks. The difference in time to plateau is thought to arise because, concurrent with the loss of viscosity, the hydrogen peroxide concentration in the gel also decreased. Although peroxide was not measured, one may expect that when the gel was spiked with large amounts of hydrogen peroxide, it will take a longer time for the concentration of H₂O₂ to decrease to a very low level at 5° C. than at 40° C. After 8 weeks, there was still an amount of H₂O₂ residue in the gel, thus, the viscosity of the gel continued to decrease.

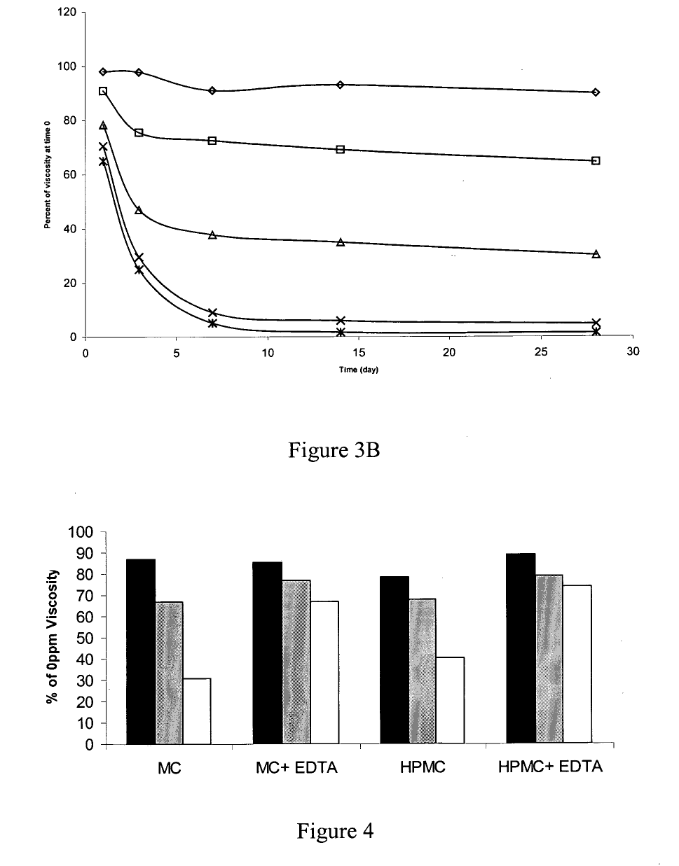

FIGS. 3A and 3B show the percent viscosity loss of MC gels over time in the presence of different levels of hydrogen peroxide at 5° C. and 40° C. FIG. 3 (Panels A and B) shows the percent of the gel viscosity remaining as compared to time zero in the presence of 0 ppm, 2 ppm, 5 ppm, 20 ppm and 50 ppm H₂O₂ at 5° C. (Panel A) and 40° C. (Panel B) for 3% methylcellulose (MC, METHOCEL® A4M) gel. In FIG. 3, ⋄=0 ppm H₂O₂, □=2 ppm H₂O₂, Δ=5 ppm H₂O₂, x=20 ppm H₂O₂ and *=50 ppm H₂O₂. Similar viscosity loss was observed in the MC gels as in the HPMC gel viscosity study. The slight increase observed in viscosity in 2 ppm hydrogen peroxide is thought to be due to experimental error. The percent error of the viscosity measurement is about ±10%.

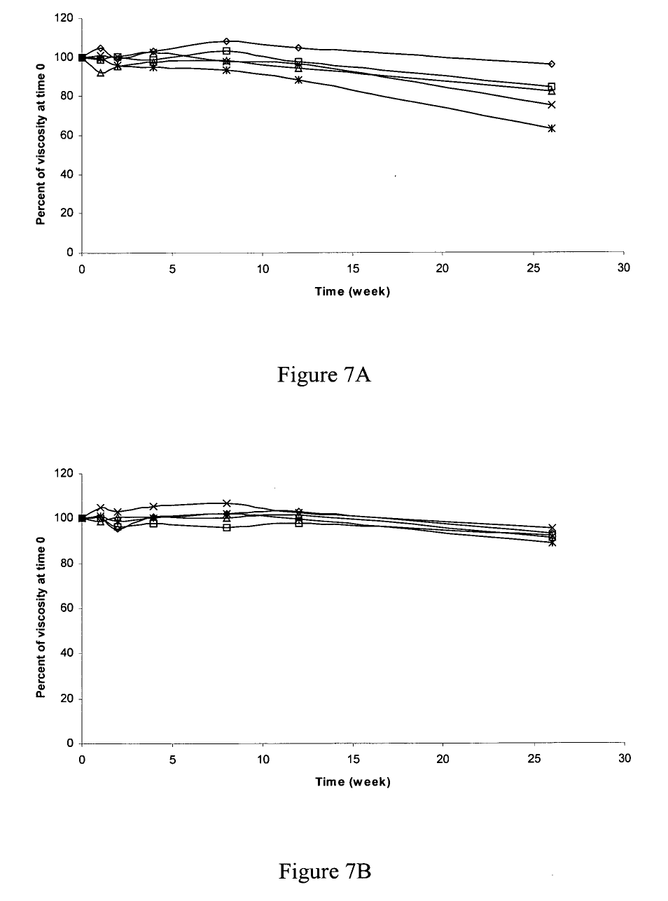

Both HPMC and MC comprise a polymeric backbone of cellulose, a natural carbohydrate that contains a basic repeating structure of anhydroglucose units. The viscosity loss of the gels from H₂O₂ was thought to be mainly due to the cleavage of the glycosidic linkage of the cellulose ether caused by hydroxyl radicals. See, e.g., McBurney, Degradation of cellulose, Cellulose and Cellulose Derivatives, Part I, Interscience, New York, 1954, p. 99. One of the mechanisms proposed of such degradation is that the C—H bonds are first oxidized, in a stepwise process, to form hydroperoxide (—C—OOH). Decomposition of the hydroperoxide formed at the acetal C—H can cleave the main polymer chain and cause a significant decrease in the gel viscosity. See, e.g., Nguyen, in Cleland J L, and Langer R, eds. Formulation and Delivery of proteins and peptides, ACS symposium series 567; 1994: 59-71, and Kuramshin, et al., J. Prakt. Chem., 1989, 331:591-599. More recently, using 1,4 anhydrocellobitol as the model compound, it has been found that the hydroxyl radicals can simply cleave any random encountered glycosidic linkages in cellulose and that the main degradation mechanism is primarily through the substitution reactions of hydroxyl radicals at the anomeric carbon, as shown in FIG. 6. See Guay, et al., J. Pulp and Paper Sci., 2002; 28(7): 217-226.

Prevention of Gel Viscosity Loss by EDTA During Autoclaving

Prior attempts to use BHA (butylated hydroxyanisole) as an anti-oxidant in HEC to prevent gel viscosity loss were not successful. See Dahl et al., 1998, Pharm. Research, 15(7): 1137-1140. While a number of anti-oxidants exist that can inhibit the oxidation of molecules, the majority of them cannot withstand the high temperatures and high pressures of the autoclave process (e.g., needed to sterilize or decrease bioburden of particular pharmaceutical compositions, etc.). Furthermore, no other such anti-oxidants have been shown to decrease/prevent viscosity loss in autoclaved hydrogels (e.g., cellulose hydrogels). As disclosed herein, EDTA and its analogues are autoclave stable and prevent/decrease autoclave/oxidative viscosity loss in cellulose and related hydrogels.

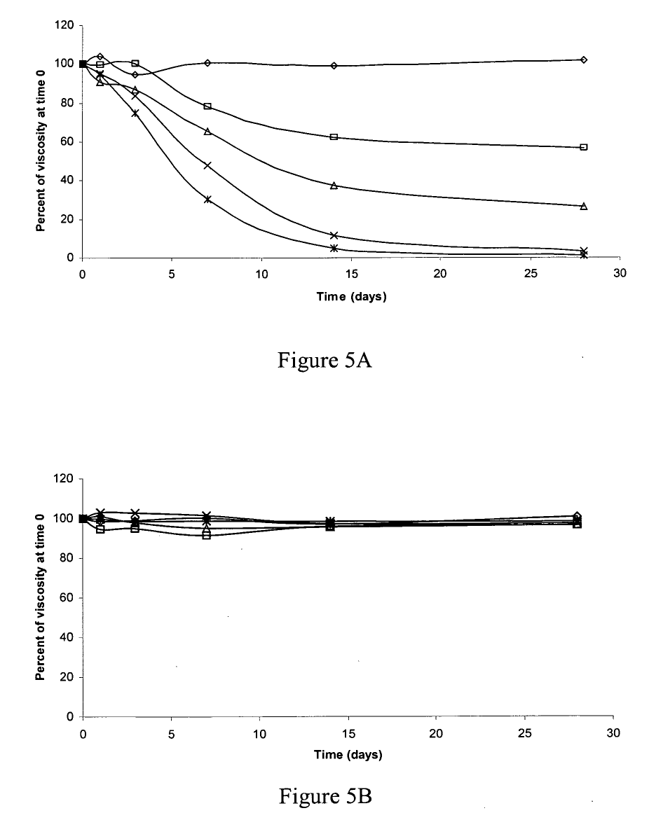

In the absence of EDTA, the loss of gel viscosity increased with the increase amount of hydrogen peroxide for both MC and HPMC as shown in FIG. 4. FIG. 4 shows the percent of the gel viscosity remaining after the autoclave process, both in the absence and presence of EDTA. In FIG. 4, the percent viscosity loss from 1 ppm H₂O₂ is indicated by solid bars, the loss from 10 ppm H₂O₂ is indicated by shaded bars, and the loss from 100 ppm H₂O₂ is indicated by unfilled bars. As can be seen, 1 ppm, 10 ppm and 100 ppm hydrogen peroxide caused gel viscosity loss to some degree. When EDTA was added to gels containing hydrogen peroxide, the viscosity loss was inhibited. The gel viscosity was well maintained in the presence of 400 ppm EDTA at hydrogen peroxide levels of 1 ppm, 10 ppm, and 1000 ppm during autoclaving. As can be seen, the benefit of EDTA was more prominent at higher concentration of H₂O₂ than at lower ones.

Effect of EDTA and Methionine on Preventing Gel Viscosity Loss During Storage

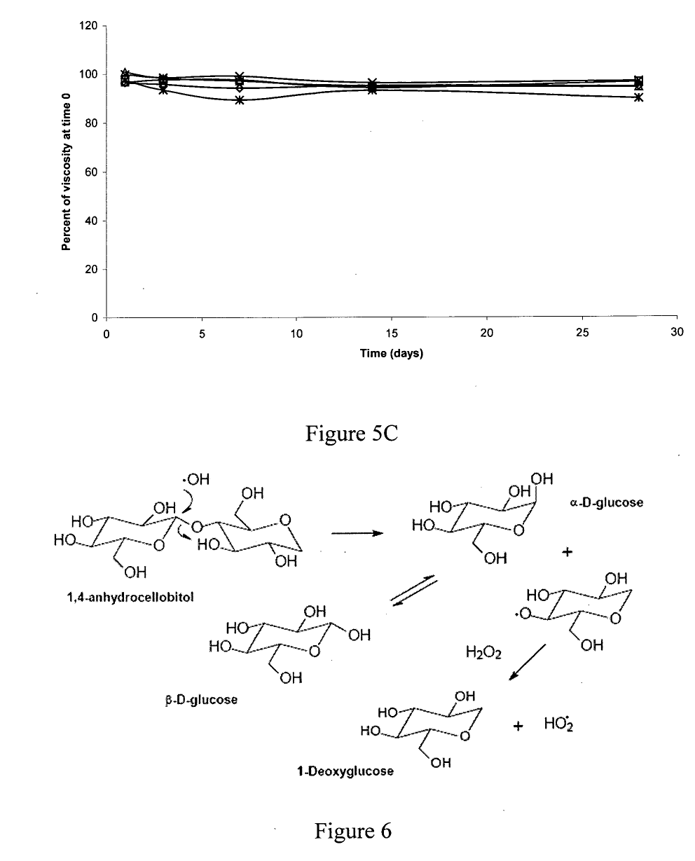

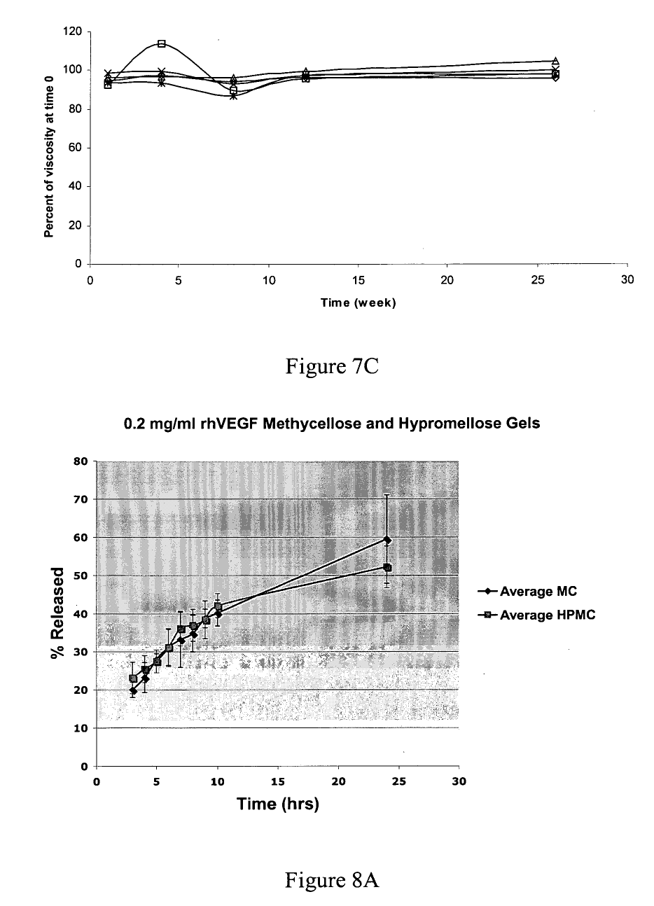

In addition to viscosity loss during autoclaving, loss of gel viscosity can also be caused by the presence of peroxides or other free radicals or oxidants during storage. As EDTA exhibited stabilization of viscosity loss caused by the autoclave sterilization process (see above), EDTA can also be used to stabilize viscosity loss during storage in the presence of added peroxide. In addition to EDTA, an antioxidant, methionine was also used as it can be added to the hydrogel after the autoclave process. Thus, the effect of EDTA and methionine on maintaining gel viscosity stability during storage time was also studied. The experiment was carried out at 40° C. for 4 weeks (FIG. 5) and at 5° C. (FIG. 7) for 6 months. FIG. 5A shows that EDTA slowed the rate of viscosity loss but did not completely prevent loss during storage at 40° C. FIG. 5 shows the percent of the viscosity of 3% HPMC (hypromellose; METHOCEL® E4M) gel in the presence of 400 ppm EDTA (Panel A), 1.8 mg/ml methionine (Panel B) and both 400 ppm EDTA and 1.8 mg/ml methionine (Panel C). The Figure compares the viscosity to that of time 0 in the presence of various concentrations of H₂O₂ at 40° C. for 4 weeks. In FIG. 5, ⋄=0 ppm H₂O₂, □=2 ppm H₂O₂, Δ=5 ppm H₂O₂, x=20 ppm H₂O₂, and *=50 ppm H₂O₂. At 40° C., the gel without addition of EDTA or methionine decreased significantly in the presence of 2 ppm, 5 ppm, 20 ppm or 50 ppm H₂O₂, respectively as discussed previously and shown in FIG. 2. FIG. 5A shows that EDTA slowed down the rate of viscosity loss compared to no EDTA (FIG. 2B). However, EDTA alone could not completely prevent viscosity loss during storage at 40° C. as shown. However, FIG. 5B shows that the addition of methionine to a final concentration of 1.8 mg/ml to the gels was sufficient to prevent/reduce gel viscosity loss during storage at 40° C. even in the presence of 50 ppm hydrogen peroxide. FIG. 5C shows that the presence of EDTA and 1.8 mg/ml methionine in a gel can prevent/reduce loss of viscosity in HPMC gels in the presence of the various concentrations of hydrogen peroxide. Overall, EDTA was shown to retard viscosity loss in the presence of peroxide during storage while methionine was shown to completely prevent viscosity loss in long term stability. Because methionine alone provided excellent protection, results in FIG. 5C cannot definitively discern the effect of combining EDTA.

Long Term Stability of Gel Viscosity in Formulations Comprising EDTA and Methionine

FIGS. 7A-7C show the stability of gel viscosity under storage at 2-8° C. in HPMC hydrogel formulations comprising EDTA, methionine, or both, in the presence of different levels of hydrogen peroxide. Comparable to the data obtained at 40° C., methionine at 5° C. effectively prevented the gel viscosity loss caused by hydrogen peroxide. However, the data in FIG. 7A indicate that EDTA also alone protected viscosity loss at 5° C. Unlike data at 40° C. (FIG. 5A) this protection at 5° C. is significant when a comparison is made between FIG. 2A (no EDTA) and FIG. 7A (with EDTA). Because protective effect of EDTA is shown, it is possible that free radical reaction is occurring. Because EDTA sequesters and inactivates the trace metals that catalyze the free radical reaction, EDTA is considered a stabilizer against free radical reactions. It is possible that at 5° C., generation of free radicals is at a slow rate and EDTA has a chance to exert the sequestering effect on the catalytic metals and, thus, minimize the amount of free radicals. Consequently, loss of viscosity could be minimized by addition of EDTA for gels stored at 5° C. Whereas, at 40° C., because free radical generation is much faster, sequestration of metal by EDTA could not reduce the formation of free radical, thus EDTA exhibited minimal effect at 40° C. FIGS. 7A through 7C show the percent of the viscosity of 3% HPMC (hypromellose; METHOCEL® E4M) gel remaining in the presence of 400 ppm EDTA (Panel A), 1.8 mg/ml methionine (Panel B), and both 400 ppm EDTA and 1.8 mg/ml methionine (Panel C) as compared to time 0 in the presence of 0 ppm (⋄), 2 ppm (□), 5 ppm (Δ), 20 ppm (x), and 50 ppm (*) H₂O₂ at 5° C. for 6 months.

FIGS. 5 and 7 show methionine to be an effective stabilizer against viscosity loss of HPMC gel when stressed by H₂O₂, while EDTA exhibits a lesser stabilization effect. Thus as can be seen, methionine can be used as part of hydrogel formulations component that are to be stored for various periods of time (e.g., hydrogel solutions such as those examined in Example 2).

As described above, and as used in the various examples herein, the use of H₂O₂ to stress a gel made of cellulose derivatives is well founded because H₂O₂ is found in various sources of cellulose raw material. See, e.g., Wasylaschuk, et al.; J. Pharm. Sci., 2007, 96:106-116. To simulate the appropriate level of peroxide to use in a stressed study, a wide range from 2 to 50 ppm of H₂O₂ was used. Based on Wasylaschuk, 2 ppm may be presented in the cellulose raw material when made into a 3% gel. Since autoclaving may generate additional free radicals and peroxide, 50 ppm may be the likely level for a gel at the initial time point of long term storage. Therefore, it is believed that 2-50 ppm is an appropriate and realistic range to use in the Examples herein. Results and recommendation based on these examples thus lead to robust and stable hydrogel formulations.

In sum, HPMC gels made from four lots of raw material were found to show viscosity loss after autoclave sterilization. A detectable viscosity loss can occur with various levels of hydrogen peroxide, e.g., 1-100 ppm, present in the HPMC and MC gels. EDTA was able to minimize the viscosity loss in the presence of hydrogen peroxide during autoclave process.

During storage at 40° C., EDTA slowed the rate of viscosity loss. At 5° C. EDTA showed further protection. Methionine was effective in completely circumventing the gel viscosity loss during storage at 5° C. or 40° C. when stressed by 2-50 ppm H₂O₂.

### Example 2

### Stabilization of Hydrogels Comprising Protein Active Agents

As described above, the current invention can optionally be used to stabilize various hydrogel formulations. Furthermore, as illustrated in this Example, methionine can be used to stabilize hydrogel formulations comprising active agents (or medicaments) such as rh VEGF. Such formulations can optionally be stored for extended periods of time (e.g., 6 months). For example, such formulations can be packaged in containers having multiple doses, thus, allowing an end user to use multiple applications of the formulation over time (e.g., over the course of treatment for a medical condition). Again, it will be appreciated that while Example 2 illustrates an exemplary embodiment of a stabilized hydrogel comprising an active agent, the particular example should not necessarily be taken as limiting. In some embodiments, the formulation is stored or packaged in, e.g., an aluminum tube, e.g., having 3.5 grams of fill at a final concentration of, e.g., 0.2, 0.6, or 1.8 mg rh VEGF per mL.

TABLE 1
Composition of stabilized rhVEGF hydrogel

| Components | Component Function | Amount per mL | |
|---|---|---|---|
| rhVEGF | Active | 0.2, 0.6, 1.8 | mg |
| Succinic acid | Buffer | 0.26 | mg |
| Succinic acid, disodium | Buffer | 0.76 | mg |
| salt, hexahydrate | | | |
| α,α-Trehalose, dihydrate | Cryoprotectant | 37.8 | mg |
| Polysorbate 20 | Surfactant | 0.036 | mg |
| Hypromellose | Viscosity enhancing agent | 30 | mg |
| (hydroxypropyl | | | |
| methylcellulose) | | | |
| Methionine-L | antioxidant | 1.8 | mg |
| Benzalkoniun chloride | Antimicrobial preservative | 0.1 | mg |
| solution | | | |
| Water for Injection | Solvent | qs to 1.0 | mL |

Table 1 illustrates a formulation, e.g., for longer term storage such as would be required for a multiple dosage package, i.e., a formulation comprising 0.2, 0.6, or 1.8 mg/mL rh VEGF; 0.26 mg/mL succinic acid; 0.76 mg/mL succinic acid, disodium salt, hexahydrate; 37.8 mg/mL α,α-Trehalose, dihydrate; 0.036 mg/mL Polysorbate 20; 30 mg/mL hypromellose (hydroxypropyl methylcellulose); 1.8 mg/mL Methionine-L; 0.1 mg/mL Benzalkoniun chloride solution; and water to qs to 1.0 mL. Thus, in this embodiment, HPMC is used as the gelling or viscosity enhancing agent, methionine as the anti-oxidant, and benzalkonium chloride as the preservative.

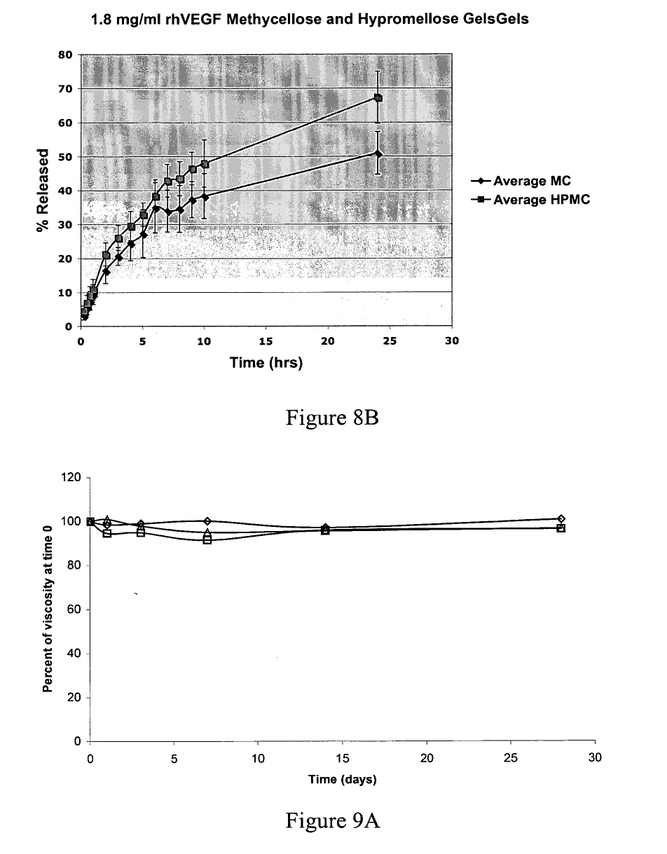

The study of the release characteristics of the present embodiment demonstrated that using HPMC as the gelling agent in such formulation did not change the release profile as shown in FIG. 8 (as compared to hydrogels comprising methylcellulose rather than HPMC). Thus, FIG. 8 shows comparison of release characteristics of a methylcellulose gel formulation and an HPMC gel formulation. As can be see from the Figure, release of telbermin was followed for up to 24 hours in Franz cells (6 replicates) where the donor and receptor chamber was separated by a semi-permeable membrane with molecular weight cut off of 1 million. Telbermin gels at two concentrations, 0.2 and 1.8 mg/mL were evaluated and the release characteristics are shown in FIG. 8, Panels A and B respectfully. As can be seen, the relative amounts of telbermin (as measured in percentages) released from 0.2 or 1.8 mg/mL were comparable.

Stabilization

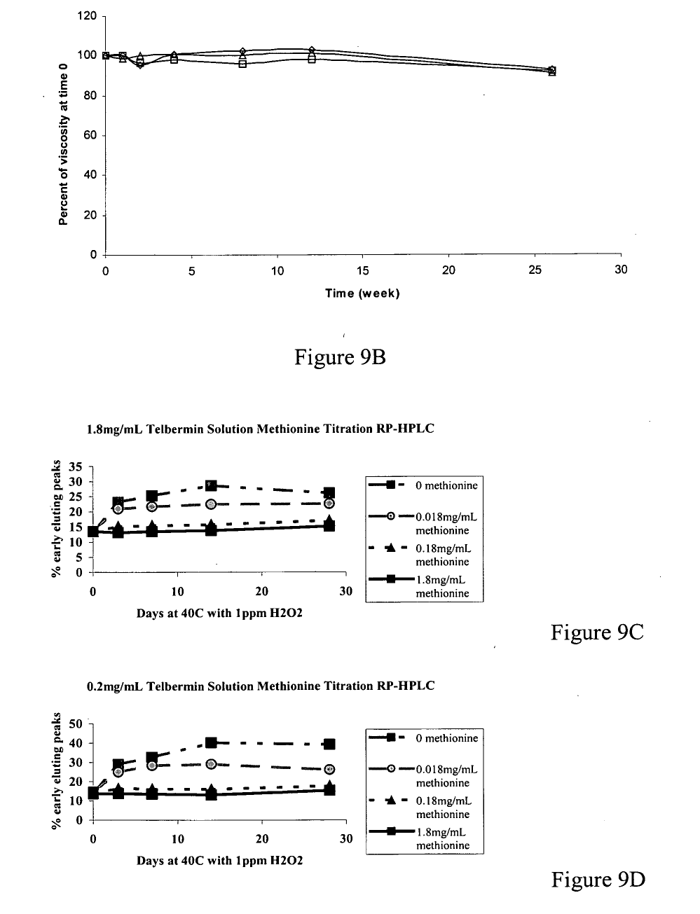

As widely cited in literature, peroxide can be found in trace levels in cellulose derivatives. Additional amounts of peroxide or free radicals can be generated when cellulose is subjected to elevated temperature such as during an autoclave sterilization process. These reactive oxygen species (peroxide and free radicals) may cause one or more effects. One is the oxidation of an active agent (such as rh VEGF), which may or may not lower the potency of the agent, another is the cleavage of the cellulosic chain which may reduce the viscosity of the drug product. Methionine, as an essential amino acid is safe to use in pharmaceutical formulations. At a concentration of 1.8 mg/mL, methionine demonstrated preservation of viscosity of the HPMC gel post autoclave sterilization. See FIG. 9 Panels A and B. In other words, the methionine, as well as the VEGF or any other temperature sensitive component, was, and can be, added to the formulation after the formulation was autoclaved. Of course, it will be appreciated that methionine's stabilization properties can also optionally be applied to hydrogels similar to that of the current Example, but which are not autoclaved. FIGS. 9A and B show the percent of the viscosity of 3% HPMC gel in the presence of 1.8 mg/ml methionine. The Figures compare the viscosity to time 0 in the presence of 0 ppm, 2 ppm, and 5 ppm H₂O₂ at 40° C. for 4 weeks (Panel A) and 5° C. for 6 months (Panel B). In FIG. 9, ⋄=0 ppm H₂O₂, □=2 ppm H₂O₂, and Δ=5 ppm H₂O₂. In addition, by using a qualitative measure of oxidized rh VEGF with HPLC, methionine effectively inhibited the oxidation of rh VEGF. See FIG. 9 Panels C and D. In FIGS. 9C and D the percent of oxidized VEGF in the presence of methionine at 0, 0.018, 0.18 and 1.8 mg/ml using rpHPLC is shown. In both viscosity and oxidation studies, small amounts of hydrogen peroxide were added to simulate the presence of reactive oxygen species. As explained above, these amounts of peroxide present in the examples herein were used to test the stabilization ability of the methods/compositions of the invention.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1.) A method of decreasing or preventing viscosity loss in a hydrogel, the method comprising: adding one or more metal ion chelators to a hydrogel solution;
autoclave sterilizing the solution; and/or, adding one or more radical scavenging moieties to the solution after the solution has been autoclave sterilized.

2.) A method of decreasing or preventing viscosity loss in a hydrogel, the method comprising: adding one or more metal ion chelators to a hydrogel solution; and/or, adding one or more radical scavenging moieties to the solution.

3.) The method of claim 1 or 2, wherein the viscosity loss results from oxidation of the hydrogel and/or from heating of the hydrogel and/or from action of free radicals upon the hydrogel.

4.) The method of claim 1 or 2, wherein the viscosity loss results from peroxide activity.

5.) The method of claim 4, wherein the peroxide comprises H₂O₂ or t-BHP.

6.) The method of claim 1 or 2, wherein the hydrogel solution comprises a plurality of polymer molecules, which molecules comprise one or more of: methyl cellulose molecules, hydroxypropyl methylcellulose molecules, or hydroxyethyl cellulose molecules.

7.) The method of claim 6, wherein the hydrogel comprises from about 1.0% to about 6.0% polymer molecule to hydrogel solution, from about 1.25% to about 5.5% polymer molecule to hydrogel solution, from about 1.5% to about 5.0% polymer molecule to hydrogel solution, from about 1.75% to about 4.5% polymer molecule to hydrogel solution, from about 2% to about 4% polymer molecule to hydrogel solution, from about 2.25% to about 4% polymer molecule to hydrogel solution, from about 2.5% to about 3.5% polymer molecule to hydrogel solution, or about 3% polymer molecule to hydrogel solution.

8.) The method of claim 1, wherein the chelator is an autoclave stable chelator.

9.) The method of claim 1 or 2, wherein the chelator is EDTA, EGTA, or PDTA.

10.) The method of claim 1 or 2, wherein the hydrogel solution comprises from about 50 to about 2000 ppm chelator, from about 100 to about 1500 ppm chelator, from about 200 to about 1000 ppm chelator, from about 300 to about 500 ppm chelator, or about 400 ppm chelator.

11.) The method of claim 1 or 2, wherein the radical scavenging moiety comprises methionine, a methionine derivative, or one or more peptides comprising methionine or a methionine derivative.

12.) The method of claim 11, wherein the hydrogel solution comprises from 0.01 to about 10 mg/ml, from about 0.05 to about 10 mg/ml, from about 0.1 to about 10 mg/ml, from about 0.15 to about 10 mg/ml, from about 0.2 to about 10 mg/ml, from about 0.5 to about 8 mg/ml, from about 1 to about 5 mg/ml, from about 1.25 to about 3 mg/ml, from about 1.5 to about 2 mg/ml, or about 1.8 mg/ml methionine.

13.) The method of claim 1 or 2, wherein the chelator comprises EDTA, wherein the scavenging moiety comprises methionine, and wherein the hydrogel solution comprises 400 ppm EDTA and 1.8 mg/ml methionine.

14.) The method of claim 1 or 2, wherein after autoclaving and/or storage, the hydrogel comprises a greater viscosity than a similar hydrogel, which similar hydrogel does not comprise the metal ion chelator and/or the radical scavenger moiety.

15.) The method of claim 14, wherein the hydrogel and the similar hydrogel are stored for an approximately equal period of time.

16.) The method of claim 15, wherein the period of time is at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 25 weeks, at least 50 weeks, at least 1 year, at least 2 years, or at least 2.25 years, at least 2.5 years, at least 2.75 years, at least 3 years at least 3.25 years, at least 3.5 years, or at least 3.75 years or longer.

17.) The method of claim 16, wherein the hydrogel and the similar hydrogel are stored at 5° C., 20-25° C., or 40° C.

18.) The method of claim 14, wherein the hydrogel comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 100%, at least 120%, at least 140%, at least 160%, at least 180%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at least 675%, at least 700%, at least 725%, at least 750%, at least 775%, at least 800%, at least 825%, at least 850%, at least 875%, at least 900%, at least 925%, at least 950%, at least 975%, or at least 1000% or more, greater viscosity as compared to the similar hydrogel.

19.) The method of claim 1 or 2, wherein the hydrogel comprises one or more pharmaceutically active agent.

20.) The method of claim 19, wherein the pharmaceutically active agent comprises vascular endothelial growth factor.

21.) A method of decreasing or preventing viscosity loss in a hydrogel, the method comprising:
providing a hydrogel solution, which solution comprises 2.5% HHX hydroxyethyl cellulose, 3.0% hydroxypropyl methylcellulose, or 3.0% methyl cellulose; adding EDTA to the hydrogel solution to a final concentration of about 400 ppm prior to autoclave sterilization of the solution; autoclave sterilizing the hydrogel solution; and, adding methionine to the autoclaved hydrogel solution to a final concentration of about 1.8 mg/ml methionine.

22.) A method of decreasing or preventing viscosity loss in a hydrogel, the method comprising:
providing a hydrogel solution, which solution comprises 30 mg/mL HPMC; 0.26 mg/mL succinic acid; 0.76 mg/mL succinic acid, disodium salt, hexahydrate; 37.8 mg/mL α,α-Trehalose, dihydrate; 0.036 mg/mL Polysorbate 20; 0.1 mg/mL Benzalkoniun chloride solution; 0.2, 0.6, or 1.8 mg/mL rh VEGF; 1.8 mg/mL Methionine-L, and water to qs to 1.0 mL; and autoclave sterilizing the hydrogel solution; further wherein the methionine and the rh VEGF are added to the hydrogel solution after autoclaving.

23.) A method of decreasing or preventing viscosity loss in a hydrogel, the method comprising:
providing a hydrogel solution, which solution comprises 30 mg/mL HPMC; 0.26 mg/mL succinic acid; 0.76 mg/mL succinic acid, disodium salt, hexahydrate; 37.8 mg/mL α,α-Trehalose, dihydrate; 0.036 mg/mL Polysorbate 20; 0.1 mg/mL Benzalkoniun chloride solution; and 0.2, 0.6, or 1.8 mg/mL rh VEGF; and adding 1.8 mg/mL Methionine-L, and water to qs to 1.0 mL to the solution.

24.) A hydrogel composition comprising a hydrogel solution, one or more metal ion chelators, and/or one or more radical scavenging moieties, which hydrogel composition has been autoclave sterilized after addition of the metal ion chelator to the solution and before addition of the radical scavenging moiety to the solution.

25.) A hydrogel composition comprising a hydrogel solution, one or more metal ion chelators, and/or one or more radical scavenging moieties.

26.) The composition of claim 24 or 25, wherein the hydrogel comprises a topical wound dressing.

27.) The composition of claim 24 or 25, wherein the hydrogel solution comprises a plurality of polymer molecules, which molecules comprise one or more of: methyl cellulose molecules, hydroxypropyl methylcellulose molecules, or hydroxyethyl cellulose molecules.

28.) The composition of claim 27, wherein the hydrogel comprises from about 1.0% to about 6.0% polymer molecule to hydrogel solution, 1.25% to about 5.5% polymer molecule to hydrogel solution, 1.5% to about 5.0% polymer molecule to hydrogel solution, 1.75% to about 4.5% polymer molecule to hydrogel solution, from about 2% to about 4% polymer molecule to hydrogel solution, from about 2.25% to about 4% polymer molecule to hydrogel solution, from about 2.5% to about 3.5% polymer molecule to hydrogel solution, or about 3% polymer molecule to hydrogel solution.

29.) The composition of claim 24 wherein the chelator is an autoclave stable chelator.

30.) The composition of claim 24 or 25 wherein the chelator is EDTA, EGTA, or PDTA.

31.) The composition of claim 24 or 25 wherein the hydrogel solution comprises from about 50 to about 2000 ppm chelator, from about 100 to about 1500 ppm, from about 200 to about 1000 ppm chelator, from about 300 to about 500 ppm chelator, or about 400 ppm chelator.

32.) The composition of claim 24 or 25 wherein the radical scavenging moiety comprises methionine, a methionine derivative, or one or more peptides comprising methionine or a methionine derivative.

33.) The composition of claim 32 wherein the hydrogel solution comprises from about 0.01 to about 10 mg/ml, from about 0.05 to about 10 mg/ml, from about 0.1 to about 10 mg/ml, from about 0.15 to about 10 mg/ml, from about 0.2 to about 10 mg/ml, from about 0.5 to about 8 mg/ml, from about 1 to about 5 mg/ml, from about 1.25 to about 3 mg/ml, from about 1.5 to about 2 mg/ml, or about 1.8 mg/ml methionine.

34.) The composition of claim 24 or 25, wherein the chelator comprises EDTA, wherein the scavenging moiety comprises methionine, and wherein the hydrogel solution comprises 400 ppm EDTA and 1.8 mg/ml methionine.

35.) The composition of claim 24 or 25, wherein after autoclaving and/or storage, the hydrogel comprises a greater viscosity than a similar hydrogel, which similar hydrogel does not comprise the metal ion chelator and/or the radical scavenger moiety.

36.) The composition of claim 35, wherein the hydrogel and the similar hydrogel are stored for an approximately equal period of time.

37.) The composition of claim 36, wherein the period of time is at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 25 weeks, at least 50 weeks, at least 1 year, at least 2 years, or at least 2.25 years, at least 2.5 years, at least 2.75 years, at least 3 years at least 3.25 years, at least 3.5 years, or at least 3.75 years or longer.

38.) The composition of claim 37, wherein the hydrogel and the similar hydrogel are stored at 5° C., 20-25° C., or 40° C.

39.) The composition of claim 35, wherein the hydrogel comprises at least 5%, at least 10%, at
least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 100%, at least 120%, at least 140%, at least 160%, at least 180%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at least 675%, at least 700%, at least 725%, at least 750%, at least 775%, at least 800%, at least 825%, at least 850%, at least 875%, at least 900%, at least 925%, at least 950%, at least 975%, or at least 1000% or more, greater viscosity as compared to the similar hydrogel.

40.) The composition of claim 24 or 25, wherein the hydrogel comprises one or more pharmaceutically active agent.

41.) The composition of claim 40, wherein the pharmaceutically active agent comprises vascular endothelial growth factor.

42.) A hydrogel composition, which composition comprises 400 ppm EDTA, which EDTA is added to the composition prior to autoclave sterilization if the hydrogel is autoclave sterilized; 1.8 mg/ml methionine, which methionine is added to the composition after autoclave sterilization if the hydrogel is autoclave sterilized; and 2.5% HHX hydroxyethyl cellulose, 3.0% hydroxypropyl methylcellulose, or 3.0% methyl cellulose.

43.) A hydrogel composition, which composition comprises hypromellose, VEGF, and methionine.

44.) The hydrogel composition of claim 43, the composition further comprising 30 mg/mL HPMC; 0.26 mg/mL succinic acid; 0.76 mg/mL succinic acid, disodium salt, hexahydrate; 37.8 mg/mL α,αx-Trehalose, dihydrate; 0.036 mg/mL Polysorbate 20; 0.1 mg/mL Benzalkoniun chloride solution; 0.2, 0.6, or 1.8 mg/ML rh VEGF; 1.8 mg/mL Methionine-L, and water to qs to 1.0 mL; and wherein if the composition has been autoclaved, the methionine and the rh VEGF have been added to the composition after autoclaving.
